(19) United States
(12) Patent Application Publication
(10) Pub. No.: US 2021/0130775 A1
(43) Pub. Date: May 6, 2021

(54) CELLS EXPRESSING A CHIMERIC ANTIGEN RECEPTOR OR ENGINEERED TCR AND COMPRISING A NUCLEOTIDE SEQUENCE WHICH IS SELECTIVELY EXPRESSED

(71) Applicant: AUTOLUS LIMITED, London (GB)

(72) Inventors: Martin Pulé, London (GB); Shaun Cordoba, London (GB); Maria Stavrou, London (GB); Vijay Peddareddigari, London (GB); Paul Smith, London (GB)

(21) Appl. No.: 16/635,740

(22) PCT Filed: Aug. 1, 2018
(86) PCT No.: PCT/GB2018/052204

§ 371 Date: Jan. 31, 2020

(30) Foreign Application Priority Data
Aug. 2, 2017 (GB) 1712407.4
Apr. 19, 2018 (GB) 1806372.7

(51) Int. Cl. C12N 5/0783; C07K 16/28; C07K 14/705; C07K 14/725; C12N 15/86; C12N 15/113

(52) U.S. Cl. CPC C12N 5/0636; C07K 16/2803; C07K 14/70517; C07K 14/7051; C07K 14/70521; C12N 2310/141; C12N 15/86; C12N 15/113; C12N 2830/005; C07K 2317/622; C07K 2319/03; C07K 14/70532

(57) ABSTRACT

The present invention provides a cell which expresses a chimeric antigen receptor (CAR) or an engineered T-cell receptor (TCR), the cell comprising a nucleotide sequence of interest (NOI) which is selectively expressed by the cell depending on: i) the differentiation/exhaustion state of the cell; or ii) the presence of an environmental metabolite in the microenvironment of the cell.

### FIELD OF THE INVENTION

The present invention relates to a cell which expresses a chimeric antigen receptor (CAR) or T-cell receptor (TCR). Expression and/or activity of the CAR or TCR can be linked to the differentiation and/or exhaustion state of the cell in which it is expressed and/or the presence of one or more environmental metabolite(s) in the microenvironment of the cell.

### BACKGROUND TO THE INVENTION

Traditionally, antigen-specific T-cells have been generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem as transgenic expression of Chimeric Antigen Receptor (CAR) allows generation of large numbers of T cells specific to any surface antigen by ex vivo viral vector transduction of a bulk population of peripheral blood T-cells.

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 2A).

The most common forms of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Clinical studies of CAR T-cells have established that CAR T-cell engraftment, expansion and persistence are a pre-requisite for clinical activity, particularly sustained responses. For example, in CD19 CAR therapy of B-cell acute lymphoblastic leukaemia failure of CAR T-cell engraftment and consequent return of the B-cell compartment is associated with relapse. Several strategies can increase the propensity of CAR T-cells to engraft, expand and persist. These include administration of preparative lymphodepleting chemotherapy, using a CAR T-cell production process which results in an increased proportion of CAR T-cells with a naive or central memory phenotype and the use of CARs with co-stimulatory signals. Despite these strategies, CAR T-cells often fail to engraft resulting in ineffective therapy.

Current CAR T-cell therapies currently typically consist of a mixture of T-cells comprising of CD4+ T-cells, CD8+ T-cells and T-cells which are naive, stem-cell memory, central memory and effector memory.

Physiologically, T-cells in different states respond differently to different signals. However, in current CAR therapies, CAR type and expression remains constant despite differentiation state and exhaustion state of the expressing T-cell. Hence as T-cells differentiate, they are currently receiving suboptimal signals.

One way of delivering optimal signals to a T-cell dependent on its phenotype is to sort the T-cells during production and transduce with different vectors. For instance, T-cells can be sorted into CD4 and CD8 populations and transduced to express CARs with different co-stimulatory signals optimized for CD4 or CD8 cells. This approach is expensive since it doubles the cell and vector production processes needed for each product. Further, for most other applications e.g. differentiation/exhaustion states—phenotypes are highly dynamic—e.g. a central memory T-cell transduced into production may remain in this compartment or may differentiate over time.

There is therefore a need for alternative approaches for the generation of CAR-expressing cells, and T-cells expressing engineered TCR, which are optimized for engraftment, expansion and persistence.

Another reason for poor persistence of CAR-T cells in vivo, particularly CAR-T cells for the treatment of solid cancers, is that the cells struggle to overcome the hostile microenvironment of the tumour. In particular, CAR T-cells may fail to engraft and expand within a solid cancer tumour bed.

There is experimental evidence for this issue, for example, mice treated with PSCA CAR-engineered T cells showed delayed tumour growth (Hillerdal et al (2014) BMC Cancer 14:30; and Abate-Daga et al (2014) 25:1003-1012). Although the cells showed high in vitro cytotoxicity, in vivo, tumour growth was delayed but tumour-bearing mice were not cured.

CAR T-cell persistence and activity can be enhanced by administration of cytokines, or by engineering the CAR T-cell to secrete or express cytokine, toxins or other factors. However, these approaches have limitations: systemic administration of cytokines can be toxic; constitutive production of cytokines may lead to uncontrolled proliferation and transformation (Nagarkatti et al (1994) PNAS 91:7638-7642; Hassuneh et al (1997) Blood 89:610-620). Expression of other factors such as transcription or survival factors are preferably expressed when the CAR T-cell is in the tumour

There is therefore a need for alternative CAR T-cell approaches, which facilitate engraftment and expansion of T cells to counteract the effects of the hostile tumour microenvironment.

### DESCRIPTION OF THE FIGURES

FIG. 1—Schematic diagram illustrating the linear model of T-cell differentiation showing the expression markers associated with each cell type. APC—antigen-presenting cell; TCM—central memory T cell; TEFF—effector T cell; TEM—effector memory T cell; TN—naive T cell; TSCM—T memory stem cell.

FIG. 2—a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 3—Schematic diagram illustrating a cassette which expresses a CAR or CAR components only in certain transcriptional states. A and B are transgenes; X is a selectively active promoter/enhancer controlling the expression of transgene A; CA is a constitutively active promoter controlling the expression of transgene B; pA is a polyadenylation sequence. X may, for example, be sensitive to T-cell exhaustion, in which case A is only expressed which the cell comprising the cassette is exhausted, whereas B is always expressed. A first specific example is where X detects exhaustion; A is an inhibitory molecule such as truncated ZAP70; and B is a CAR. When the cassette is expressed in a T-cell, the inhibitory molecule is only expressed when the T-cell is exhausted to prevent further exhaustion and dampen down CAR activity. A second specific example is where X detects differentiation to effector memory; A is a CAR with a 41 BB-Z endodomain; and B is a CAR with a CD28-Z endodomain. When the cassette is expressed in a T-cell, only the CD28-Z CAR is expressed while the cells is in the naï/central memory state. When the cell differentiates to effector memory the 41 BB-Z CAR is also expressed causing rapid expansion.

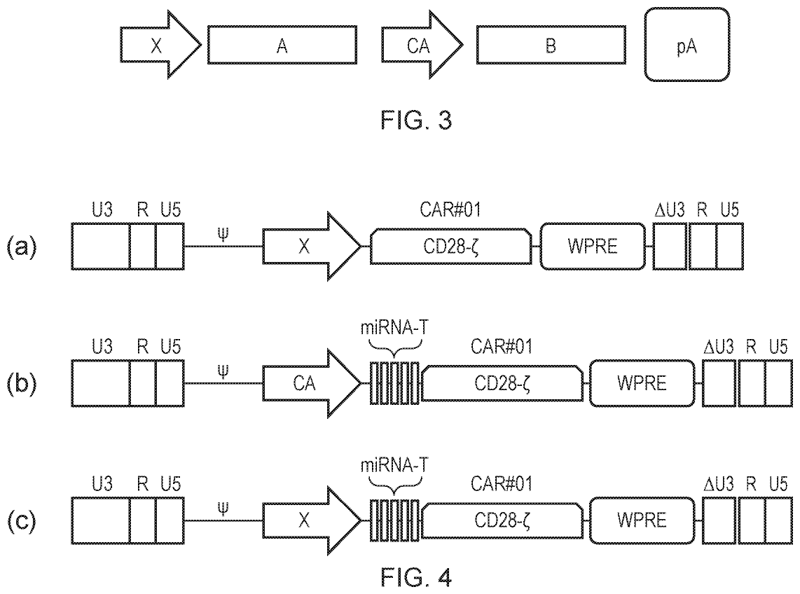

FIG. 4—Schematic diagram to illustrate the different ways in which a single transgene can be selectively expressed. (a) A self-inactivating retroviral vector is shown with an internal promoter ‘X’ which drives transcription only in a particular T-cell context. In this case, CAR-01 will only be expressed when promoter X is active. Retroviral long-terminal repeat U3, R and U5 regions are shown along with Packaging signal—yf and the woodchuck pre-processing element WPRE. (b) Alternatively, gene-expression can be under the control of a constitutively active promoter (CA). In this case, control of protein expression is achieved by incorporating specific miRNA target sequence in the 5′ untranslated region of the transcript. In T-cell contexts where the miRNA is expressed, the transcript will be degraded. (c) In some applications, both methods are applied.

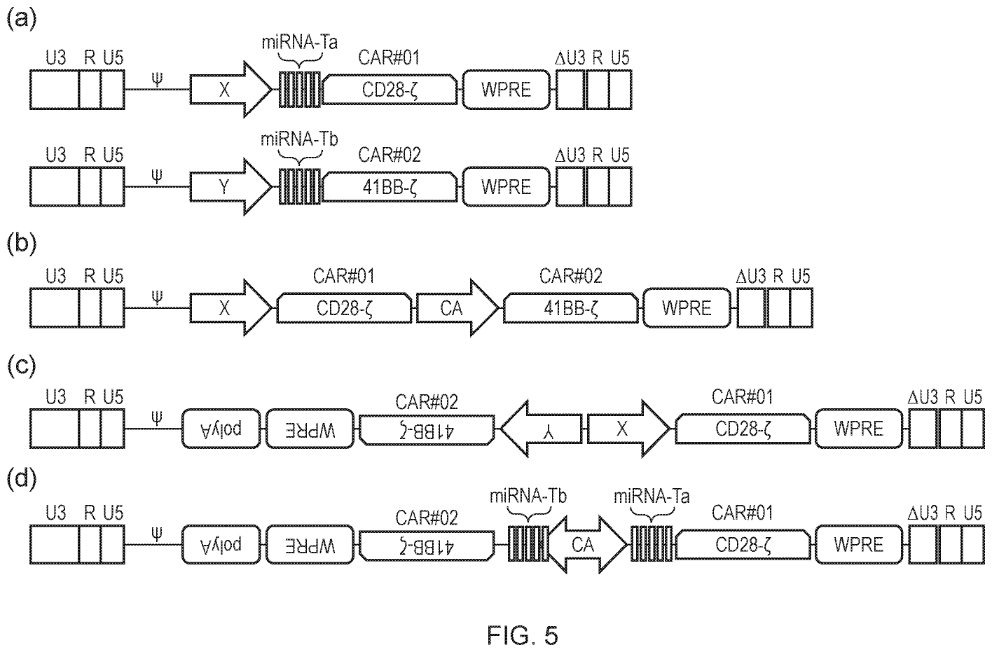

FIG. 5—Schematic diagram illustrating strategies for having independently expressed transgenes (a) Two separate cassettes controlled by either or both specific promotors/miRNA target sequences are introduced simultaneously into a T-cell. (b) Expression cassettes can be engineered to incorporate split transcriptional systems. One method is to have a vector express two transcripts. A 5′ selectively active promoter drives transcription of a long transcript where the first open reading frame codes for a first protein which should be selectively expression. Downstream from this, a second constitutively active promoter in the same orientation as the first drives transcription of a shorter transcript where a second open reading frame codes for a second protein which should be constitutively expressed. Both transcripts share the same polyA adenylation signal. (c) Alternatively, two separate promoters can drive expression of two independent transcripts. This is most conveniently achieved by orientating the transcripts head-to-head with one transcript read from the sense strand and the other read from the anti-sense strand. (d) As a further alternative, a constituitively active bi-directional promoter results in transcription of two transcripts in opposite direction. Each transcript is controlled by a separate miRNA target sequence.

FIG. 6—Schematic diagram illustrating the Aryl Hydrocarbon Receptor (AHR) pathway

FIG. 7—Schematic diagram illustrating the kynurenine pathway

FIG. 8—Schematic diagram illustrating structure of Aryl Hydrocarbon Receptor (AHR)

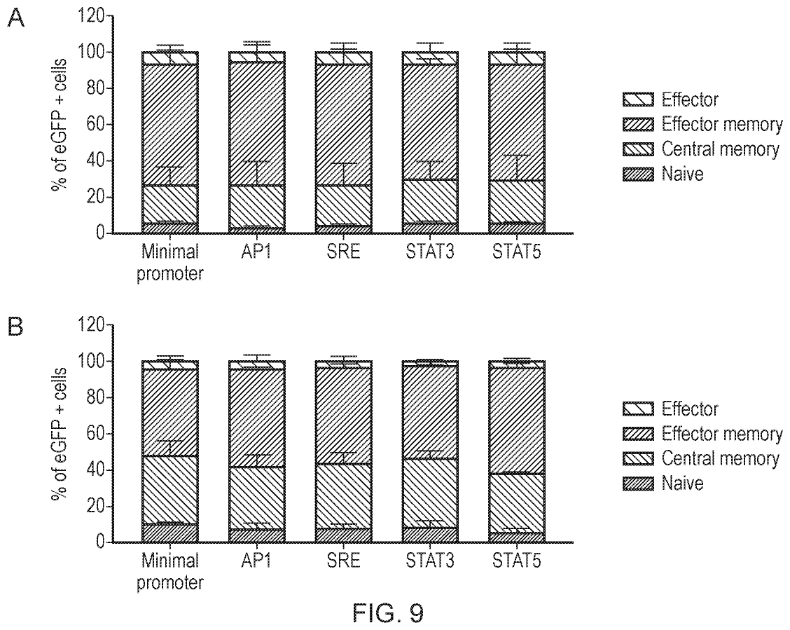

FIG. 9—Memory phenotype of T cells expressing reporter gene under the control of various different promoter at 72 hours A) without stimulation, and B) stimulated with 3 ug/mL PHA and 50 IL-2 U/mL.

FIG. 10—Differential expression of reporter gene eGFP in different memory subsets of transduced T cells in which the reporter gene is under the control of various different promoters, at 72 hours A) without stimulation, and B) stimulated with 3 ug/mL PHA and 50 IL-2 U/ml

FIG. 11—Flow cytometric analysis of eGFP expression in different memory subsets of transduced T cells in which the reporter gene is under the control of a CREB-responsive promoter at 24h hours either with or without PHA stimulation

FIG. 12—A) Memory phenotype of T cells expressing reporter gene under the control of a CREB-responsive promoter at 24h hours either with or without PHA stimulation; B) Differential expression of reporter gene eGFP in different memory subsets of transduced T cells in which the reporter gene is under the control of a CREB-responsive promoter at 24h hours either with or without PHA stimulation.

### SUMMARY OF ASPECTS OF THE INVENTION

The present inventors have found that it is possible to optimise the function of CAR-expressing or TCR-expressing cells by tailoring the expression of, for example the CAR/TCR, a CAR component or an agent which modulates CAR/TCR activity to the transcriptional state of the cell. Expression of one or more genes can be linked to the differentiation or exhaustion state of the cell, meaning that the structure of the CAR or CAR activity can be controlled over time.

This technology has many applications, including skewing CAR-expressing cells towards a more ‘naï’ state to improve their efficacy and survival in patients.

It is also possible to control the timing and/or location in vivo of CAR/TCR expression and/or CAR/TCR cell activity by tailoring the expression of, for example, the CAR/TCR, a CAR component or an agent which modulates CAR/TCR activity to the presence of an environmental metabolite in the microenvironment of the CAR/TCR expressing cell

Thus in a first aspect, the present invention provides a cell which expresses a chimeric antigen receptor (CAR) or engineered T-cell receptor (TCR), the cell comprising a nucleotide sequence of interest (NOI) which is selectively expressed depending on:
i) the differentiation/exhaustion state of the cell; or
ii) the presence of an environmental metabolite in the microenvironment of the cell.

In a first embodiment of the first aspect of the invention, the NOI is selectively expressed depending on the differentiation and/or exhaustion state of the cell.

The NOI may be selectively expressed in, for example, a CD4+ T cell, a CD8+ T cell, a regulatory T cell, a naive T cell, a central memory T cell, an effector memory T cell, an effector T cell, or an exhausted T cell.

Expression of the NOI may be under the control of a selectively active promoter.

The cell may comprise an miRNA target sequence such that expression of the NOI in the cell is controlled by an miRNA.

Expression of the NOI in the cell may be under the control of a selectively active promoter and an miRNA target sequence.

In a second embodiment of the first aspect of the invention, the NOI is selectively expressed depending the presence of an environmental metabolite in the microenvironment of the cell

The environmental metabolite may activate the aryl hydrocarbon receptor (AHR).

The environmental metabolite is a tryptophan metabolite such as is kynurenine.

For both the first and second embodiments of the cell of the first aspect of the invention, the NOI may encode a chimeric antigen receptor (CAR) or an engineered T-cell receptor.

Alternatively the NOI may encode a CAR component such as a receptor component or an intracellular signalling component.

The NOI may encode an agent which modulates CAR or TCR activity such as, for example, a signal transduction modifying protein, a dampener; an inhibitory CAR, a cytokine signalling domain, an adhesion molecule or a transcription factor.

The NOI may encode an agent which modulates activity of the cell, for example a cytokine, an adhesion molecule or a transcription factor.

The NOI may encode an agent which modulates activity of the target cell. For example, the agent may comprise a toxin.

The NOI may encode an agent which modulates the target cell microenvironment. For example, the agent may be a chemokine or a cytokine, or an agent which affects cytokine or chemokine-mediated signalling such as a dominant negative chemokine/cytokine or chemokine/cytokine receptor or a binding agent, such as an antibody or antibody fragment which modulates chemokine/cytokine-mediated signalling.

In a second aspect, the present invention provides a nucleic acid sequence.

In a first embodiment of the second aspect of the invention there is provided a nucleic acid sequence which comprises a nucleotide sequence of interest (NOI) which is selectively active depending on the differentiation/exhaustion state of the cell in which it is expressed.

The NOI may be under the control of a promoter which is selectively active depending on the differentiation/exhaustion state of the cell in which it is expressed.

Alternatively, or in addition, the NOI may comprise a specific miRNA target sequence which causes transcript degradation at a certain differentiation/exhaustion state of the cell in which the nucleic acid sequence is expressed.

In a second embodiment of the second aspect of the invention, there is provided a nucleic acid sequence which comprises a nucleotide sequence of interest (NOI) under the control of a promoter which is selectively active depending the presence of an environmental metabolite in the microenvironment of the cell in which it is expressed.

In a third aspect the present invention provides a kit of nucleic acid sequences which comprises a nucleic acid sequence according to the second aspect of the invention.

The kit may comprise:
(i) a first nucleic acid sequence under the control of a constitutively active promoter; and
(ii) a second nucleic acid sequence under the control of a promoter which is selectively active depending on either:

the differentiation/exhaustion state of the cell in which it is expressed or the presence of an environmental metabolite in the microenvironment of the cell in which it is expressed.

The kit may comprise a first nucleic acid sequence under the control of a first selectively active promoter; and second nucleic acid sequence under the control of a second selectively active promoter wherein the first and second promoters are active at different differentiation/exhaustion states of the cell in which the kit of nucleic acid sequences is expressed.

The kit may comprise:
(i) a first nucleic acid sequence which comprises a specific miRNA target sequence which causes transcript degradation at a certain differentiation/exhaustion state of the cell in which the nucleic acid sequence is expressed; and
(ii) a second nucleic acid sequence which lacks a specific miRNA target sequence.

The kit may comprise a first nucleic acid sequence having a first miRNA target sequence; and second nucleic acid sequence having a second miRNA target sequence wherein the first and second miRNA target sequences causes transcript degradation at different differentiation/exhaustion states of the cell in which the kit of nucleic acid sequences is expressed.

In a fourth aspect, the present invention provides a nucleic acid construct which comprises a nucleic acid sequence according to the second aspect of the invention.

The nucleic acid construct may comprise:
(i) a first nucleic acid sequence under the control of a constitutively active promoter; and
(ii) a second nucleic acid sequence under the control of a promoter which is selectively active depending on either the differentiation/exhaustion state of the cell in which it is expressed or the presence of an environmental metabolite in the microenvironment of the cell in which it is expressed.

The nucleic acid construct may comprise a first nucleic acid sequence under the control of a first selectively active promoter; and second nucleic acid sequence under the control of a second selectively active promoter wherein the first and second promoters are active at different differentiation/exhaustion states of the cell in which the nucleic acid construct is expressed.

The nucleic acid construct may comprise:
(i) a first nucleic acid sequence which comprises a specific miRNA target sequence which causes transcript degradation at a certain differentiation/exhaustion state of the cell in which the nucleic acid construct is expressed; and
(ii) a second nucleic acid sequence which lacks a specific miRNA target sequence.

The nucleic acid construct may comprise a first nucleic acid sequence having a first miRNA target sequence; and second nucleic acid sequence having a second miRNA target sequence wherein the first and second miRNA target sequences causes transcript degradation at different differentiation/exhaustion states of the cell in which the nucleic acid construct is expressed.

The first and second nucleic acid sequences may be under the control of a constitutively active bi-directional promoter.

The first nucleic acid sequence may encode a chimeric antigen receptor (CAR), CAR component or engineered T-cell receptor (TCR) and the second nucleic acid sequence may encode an inhibitory molecule, such that when the nucleic acid construct is expressed in a T cell, the CAR or CAR component or TCR is expressed constitutively, but the inhibitory molecule is selectively expressed when the T cell is exhausted, the inhibitory molecule causing a reduction in CAR or TCR activity.

The inhibitory molecule may, for example, comprise truncated ZAP70 which comprises one or more ITAM-binding domain(s) but lacks a kinase domain.

The first nucleic acid sequence may encode a CAR or CAR component comprising a CD28 co-stimulatory domain; and the second nucleic acid sequence may encode a CAR or CAR component comprising an OX40 or 41 BB co-stimulatory domain, such that when the nucleic acid construct is expressed in a T cell, the first CAR or CAR component is expressed constitutively, but the second CAR or CAR component is selectively expressed when the cell is in an effector memory or effector state.

The first nucleic acid sequence may encode a chimeric antigen receptor (CAR), CAR component or engineered T cell receptor (TCR) and the second nucleic acid sequence may encode a cytokine, such that when the nucleic acid construct is expressed in a T cell, the CAR, CAR component or TCR is expressed constitutively, but the cytokine is selectively expressed in the presence of an environmental metabolite in the microenvironment of the T cell.

In a fifth aspect, the present invention provides a vector which comprises a nucleic acid sequence according to the second aspect of the invention; a kit of nucleic acid sequences according to the third aspect of the invention; or a nucleic acid construct according to the fourth aspect of the invention.

In a sixth aspect, the present invention provides a method for making a cell according to the first aspect of the invention which comprises the step of introducing: a nucleic acid sequence according to the second aspect of the invention; a kit of nucleic acid sequences according to the third aspect of the invention; or a nucleic acid construct according to the fourth aspect of the invention; or a vector according to fifth aspect of the invention into a cell.

The cell may be from a sample isolated from a subject.

In a seventh aspect, the present invention provides a pharmaceutical composition comprising a plurality of cells according to the first aspect of the invention.

In an eighth aspect, the present invention provides a pharmaceutical composition according to the seventh aspect of the invention for use in treating and/or preventing a disease.

In a ninth aspect, the present invention provides a method for treating and/or preventing a disease, which comprises the step of administering a pharmaceutical composition according to the seventh aspect of the invention to a subject.

The method may comprise the following steps:
(i) isolation of a cell-containing sample;
(ii) transduction or transfection of the cells with : a nucleic acid sequence according to the second aspect of the invention; a kit of nucleic acid sequences according to the third aspect of the invention; or a nucleic acid construct according to the fourth aspect of the invention; or a vector according to fifth aspect of the invention; and
(iii) administering the cells from (ii) to a subject.

In a tenth aspect, the present invention provides the use of a pharmaceutical composition according to the seventh aspect of the invention in the manufacture of a medicament for the treatment and/or prevention of a disease.

The disease may be cancer.

### DETAILED DESCRIPTION

The present invention provides a cell which comprises a nucleotide of interest (NOI) which is selectively expressed depending on the transcriptional state of the cell or the presence of an environmental metabolite in the microenvironment of the cell.

The NOI may, for example, be selectively expressed at a certain differentiation or exhaustion state of the cell.

The cell may be a T cell.

T Cell Differentiation

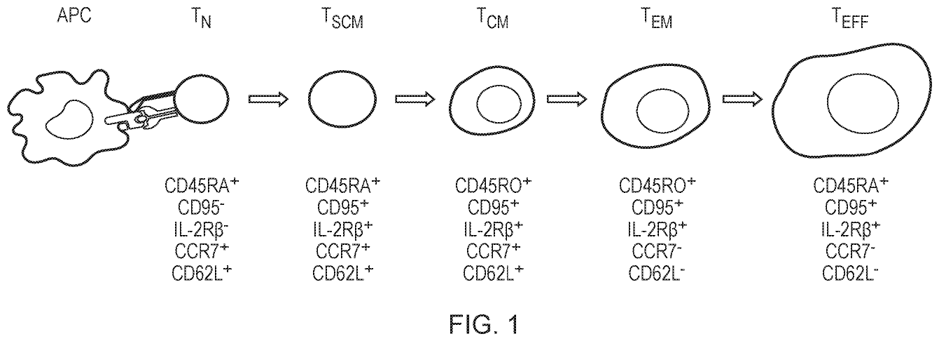

Following activation, T-cells differentiate into a variety of different T-cell subtypes, as shown in FIG. 1.

T cell differentiation and memory and effector T cells play a significant role in immunity against pathogenic agents. When an antigen-presenting cell presents a pathogenic antigen to naive T cells, the cells become activated, increase in cell number, and differentiate into effector cells which migrate to the site of infection and eliminate the pathogen. The effector cells are short-lived cells, while the subset of memory cells which is formed has a potential of long-term survival. Memory cells can be located in the secondary lymphoid organs (central memory cells, T CM) or in the recently infected tissues (effector memory cells, T EM cells). During re-exposure to antigen during a secondary immune response, memory T cells undergo fast expansion and cause a more effective and faster immune response compared the primary immune response in eliminating infection. Memory cells have several characteristic features: i) the presence of previous expansion and activation; (ii) persistence in the absence of antigen; and iii) increased activity upon re-exposure to antigen.

Distinct T cell subsets, or distinct T-cell differentiation states, can be identified based on the cell surface markers expressed and/or the effector molecules they produce. The following tables summarise various T cell subsets based in terms of their surface phenotype, transcriptional regulators, effector molecules and function in immune responses.

To connect transgenic transcription to a particular T-cell state, a promoter from a selective surface marker can be used to drive transgenic transcription. Alternatively, a transcriptional element responsive to a transcription factor selective for that state can be used.

Naïve T Cells

1. CD4+ naïve T cell

| | |
|---|---|
| Surface phenotype | TCR, CD3, CD4, CCR7, CD62Lhi, IL-7R |
| | (CD127) |
| Transcription factors | THPOK |
| Function | Patrol through lymph nodes scanning |
| | peptide-MHC class II molecule |
| | complexes on APCs for the |
| | presence of their cognate antigen. |
| | Following activation by APCs, naive |
| | CD4+ T cells differentiate into |
| | effector or regulatory T cells; |
| | activated naive T cells also give |
| | rise to memory T cells |
| Other features | CD45RA expressed by human cells. |

2. CD8+ naïve cell

| | |
|---|---|
| Surface phenotype | TOR, CD3, CD8, CCR7, CD62Lhi, IL-7R |
| | (CD127) |
| Transcription factors | RUNX3 |
| Function | Patrol through lymph nodes scanning |
| | peptide-MHC class I molecule |
| | complexes for the presence of their |
| | cognate antigen. Following activation |
| | by APCs, they differentiate |
| | into CTLs and memory T cells. |
| Other features | CD45RA expressed by human cells |

Central Memory T Cells

| | |
|---|---|
| Surface phenotype | CCR7hi, CD44, CD62Lhi, TCR, CD3, |
| | IL-7R (CD127), IL-15R |
| Transcription factors | BCL-6, BCL-6B, MBD2, BMI1 |
| Effector molecules secreted | IL-2, CD40L Low levels IL-4, |
| | IFNγ, IL-17A |
| Function | Preferentially reside in secondary |
| | lymphoid organs, mounting recall |
| | responses to antigens. Even though |
| | these cells lack immediate effector |
| | functions, they rapidly proliferate |
| | and differentiate into effector T |
| | cells following antigen stimulation. |

Effector memory T cells

| | |
|---|---|
| Surface phenotype | CD62Llow, CD44, TCR, CD3, IL-7R |
| | (CD127), IL-15R, CCR7low |
| Transcription factors | BLIMP1 |
| Effector molecules secreted | Rapid and high production of |
| | inflammatory cytokines |
| Function | Preferentially found in peripheral |
| | tissues. They provide immediate |
| | protection upon antigen challenge |
| | through, for example, the rapid |
| | production of effector cytokines. |

Effector T Cells

1. Cytotoxic T cell (CTL)

| | |
|---|---|
| Surface phenotype | TCR, CD3, CD8 |
| Transcription factors | EOMES, T-bet, BLIMP1 |
| Effector molecules secreted | Perforin, granzyme, IFNγ |
| Function | Cytotoxic; kill infected and |
| | transformed cells and thereby |
| | protect the host from viral |
| | infections and cancer. Direct |
| | killing is mediated by secretion |
| | of perforin and granzymes, which |
| | cause apoptosis of target cells. |
| Other features | In humans, mainly CD45RO+. Some |
| | terminally differentiated CTLs |
| | in humans re-express CD45RA |

2. T_{H}1 cell

| | |
|---|---|
| Surface phenotype | TCR, CD3, CD4, IL-12R, IFNγR, |
| | CXCR3 |
| Transcription factors | T-bet, STAT4, STAT1 |
| Effector molecules secreted | IFNγ, IL-2, LTα |
| Function | Promote protective immunity |
| | against intracellular pathogens. |
| | By secreting IFNγ, they induce |
| | activation of macrophages and |
| | upregulation of iNOS, leading |
| | to the killing of intracellular |
| | pathogens such as Leishmania |
| | major, Listeria monocytogenes |
| | and Mycobacterium spp. Their |
| | development is regulated by |
| | IL-12. |

3. T_{H}2 cell

| | |
|---|---|
| Surface phenotype | TCR, CD3, CD4, IL-4R, IL-33R, |
| | CCR4, IL-17RB, CRTH2 |
| Transcription factors | GATA3, STAT6, DEC2, MAF |
| Effector molecules secreted | IL-4, IL-5, IL-13, IL-10 |
| Function | Promote humoral immune responses |
| | and host defence against |
| | extracellular parasites. However, |
| | they can also potentiate allergic |
| | responses and asthma. Their |
| | development and maintenance is |
| | regulated by IL-4, IL-25 and IL- |
| | 33. |
| Other features | IRF4 is also an important |
| | transcription factor. |

4. T_{H}9 cell

| | |
|---|---|
| Surface phenotype | TOR, CD3, CD4 |
| Transcription factors | PU.1 |
| Effector molecules secreted | IL-9, IL-10 |
| Function | Involved in host defence against |
| | extracellular parasites, primarily |
| | nematodes. Despite their production |
| | of anti-inflammatory IL-10, they |
| | promote allergic inflammation. |

5. T_{H}17 cell

| | |
|---|---|
| Surface phenotype | TCR, CD3, CD4, IL-23R, CCR6, |
| | IL-1R, CD161 (human only) |
| Transcription factors | RORγt, STAT3, RORα |
| Effector molecules secreted | IL-17A, IL-17F, IL-21, IL-22, |
| | CCL20 |
| Function | Promote protective immunity |
| | against extracellular bacteria |
| | and fungi, mainly at mucosal |
| | surfaces. Also promote autoimmune |
| | and inflammatory diseases. |
| | Generated in the presence of TGFβ |
| | and IL-6 and/or IL-21 and are |
| | maintained by IL-23 and IL-1. |
| Other features | Also express BATF, IκBζ, IRF4 and |
| | AHR transcription factors. Human |
| | T_{H}17 cellsalso produce IL-26. |

6. T_{H}22 cell

| | |
|---|---|
| Surface phenotype | TCR, CD3, CD4, CCR10 |
| Transcription factors | AHR |
| Effector molecules secreted | IL-22 |
| Function | Identified in inflammatory skin |
| | diseases. |

7. T_{FH} cell

| | |
|---|---|
| Surface phenotype | TCR, CD3, CD4, CXCR5, SLAM, |
| | OX40L, CD40L, ICOS, IL-21R, PD1 |
| Transcription factors | BCL-6, STAT3 |
| Effector molecules secreted | IL-21 |
| Function | These cells are involved in |
| | promotion of germinal centre |
| | responses and provide help for B |
| | cell class switching. |
| Other features | SAP expression |

8. Natural T_{Reg} cell

| | |
|---|---|
| Surface phenotype | TCR, CD3, CD4, CD25, CTLA4, GITR |
| Transcription factors | FOXP3, STAT5, FOXO1, FOXO3 |
| Effector molecules secreted | IL-10, TGFβ, IL-35 |
| Function | Mediate immunosuppression and |
| | tolerogenic responses through |
| | contact-dependent and independent |
| | mechanisms. These cells are - |
| | generated in the thymus. |

9. Inducible T_{Reg} cell

| | |
|---|---|
| Surface phenotype | TCR, CD3, CD4, CD25, CTLA4, GITR |
| Transcription factors | FOXP3, FOXO1, FOXO3, STAT5, SMAD2, |
| | SMAD3, SMAD4 |
| Effector molecules secreted | IL-10, TGFβ |
| Function | Promote immunosuppression and |
| | tolerance by contact-dependent |
| | and -independent mechanisms. |
| | These cells are generated from |
| | naive T cells in the periphery |
| | and, at least in some cases, |
| | TGFβ and IL-2 are important for |
| | their differentiation. |

10. T_{R}1 cell

| | |
|---|---|
| Surface phenotype | TCR, CD3, CD4 |
| Effector molecules secreted | IL-10 |
| Function | Immunosuppression mediated by |
| | IL-10 production. These cells |
| | are generated from naive T cells |
| | in the presence of TGFβ and IL- |
| | 27 or in the presence of the |
| | immunosuppressive drugs vitamin |
| | D3 and dexamethasone. |

In the context of the present invention, the NOI may be selectively expressed in:
a) a naïT cell;
b) a CD4+ T cell;
c) a CD8+ T cell;
d) a central memory T cell;
e) an effector memory T cell;
f) a regulatory T cell; or
g) an effector T cell.

The NOI may be under the control of a promoter which causes selective expression in a particular T cell subset. For example, the NOI may be under the control of an AP1-, CREB-, SRE-, TCF-LEF-, STAT3-, or STATS-responsive promoter.

The sequences of these promoters are shown below as SEQ ID No. 27 to 32.

(AP1-responsive promoter)
SEQ ID No. 27
TGAGTCAGTGACTCAGTGAGTCAGTGACTCAGTGAGTCAGTGACTCAG
(CREB-responsive promoter)
SEQ ID No. 28
GCACCAGACAGTGACGTCAGCTGCCAGATCCCATGGCCGTCATACTGTGA
CGTCTTTCAGACACCCCATTGACGTCAATGGGAGAAC
(SRE-responsive promoter)
SEQ ID No. 29
AGGATGTCCATATTAGGACATCTAGGATGTCCATATTAGGACATCTAGGA
TGTCCATATTAGGACATCTAGGATGTCCATATTAGGACATCTAGGATGTC
CATATTAGGACATCT
(TCF-LEF-responsive promoter)
SEQ ID No. 30
AGATCAAAGGGTTTAAGATCAAAGGGCTTAAGATCAAAGGGTATAAGATC
AAAGGGCCTAAGATCAAAGGGACTAAGATCAAAGGGTTTAAGATCAAAGG
GCTTAAGATCAAAGGGCCTA
(STAT3-responsive promoter)
SEQ ID No. 31
AGCTTCATTTCCCGTAAATCGTCGAAGCTTCATTTCCCGTAAATCGTCGA
AGCTTCATTTCCCGTAAATCGTCGAAGCTTCATTTCCCGTAAATCGTCGA
AGCTTCATTTCCCGTAAATCGTCGA
(STAT5-responsive promoter)
SEQ ID No. 32
AGTTCTGAGAAAAGTAGTTCTGAGAAAAGTAGTTCTGAGAAAAGTAGTTC
TGAGAAAAGTAGTTCTGAGAAAAGT

T Cell Exhaustion

T cell exhaustion is a state of T cell dysfunction that arises during many chronic infections and cancer. It is defined by poor effector function, sustained expression of inhibitory receptors and a transcriptional state distinct from that of functional effector or memory T cells.

Both extrinsic negative regulatory pathways (such as immunoregulatory cytokines) and cell intrinsic negative regulatory pathways (such as PD-1) have key roles in exhaustion. Exhausted T cells represent a distinct state of T cell differentiation.

Exhausted CD8+ T cells were first identified during chronic viral infection as virus-specific, tetramer-positive CD8+ T cells that do not produce cytokines. During exhaustion, loss of function occurs in a hierarchical manner, with exhausted CD8+ T cells losing some properties before losing others. Typically, functions such as IL-2 production, high proliferative capacity and ex vivo killing are lost first. Other properties, including the ability to produce tumor necrosis factor, are often lost at more intermediate stages of dysfunction. Severe exhaustion eventually leads to virus-specific cells that partially or, in some cases, completely lack the ability to produce large amounts of interferon-γ (IFN-γ) or beta-chemokines or to degranulate. The final stage of exhaustion is physical deletion of virus-specific T cells. Virus-specific CD4+ T cells also lose effector function during chronic viral infection.

Immunoregulation is centrally involved in T cell exhaustion. These negative pathways can be grouped into three main categories: cell surface inhibitory receptors (such as PD-1), soluble factors (such as IL-10), and immunoregulatory cell types (such as regulatory T cells (Treg cells) and other cells).

Several specific transcriptional pathways have been implicated in T cell exhaustion. For example, the transcriptional repressor Blimp-1 is centrally involved in CD8+T cell exhaustion. Transcriptional profiling indicates higher expression of the transcription factor NFATc1 (NFAT2) in exhausted CD8+ T cells.

An integrated genomics approach has been used to define genes that are induced by PD-1 ligation and also involved in T cell exhaustion in mice and in humans. Such studies have identified BATF as a common transcriptional pathway downstream of PD-1 in exhausted T cells. BATF forms dimers with the transcription factor c-Jun, displacing the transcription factor c-Fos, and can inhibit canonical AP-1-mediated transcription.

The following table summarises defines exhausted T cells in terms of their surface phenotype, transcriptional regulators, effector molecules and function in immune responses.

| | |
|---|---|
| Surface phenotype | CD3, CD8, PD1, TIM3, 1B11, LAG3 |
| Transcription | BLIMP-1 |
| factors | |
| Function | Generated in response to chronic |
| | antigen mediated TCR stimulation. |
| | These cells express inhibitory |
| | receptors and lack effector |
| | cytokine production; they |
| | therefore fail to mount effective |
| | antiviral immune responses. |

In the context of the present invention, the NOI may be selectively expressed in an exhausted T cell. To achieve this transgenic transcription can be driven by promoters taken from markers of exhaustion such as PD1, TIM3 and Lag3.

Selectively Active Promoters

The term “promoter” used herein means a promoter and/or enhancer. A promoter is a region of DNA that initiates transcription of a particular gene. Promoters are located near the transcription start sites of genes, on the same strand and upstream on the DNA (towards the 5′ region of the sense strand). Promoters are usually about 100-1000 base pairs long. An enhancer is a short (50-1500 bp) region of DNA that can be bound by transcription factors to increase the likelihood that transcription of a particular gene will occur. Enhancers are cis-acting and can be located upstream or downstream from the transcription start site.

Expression of a transgene can be restricted to a particular differentiation state of a T-cell by means of specific promotors which physiologically directs expression of a transgene in said T-cell state. For instance, expression of a transgene can be linked to differentiation of a T-cell to a CD4+cell by driving expression of said transgene from a CD4 promoter. Expression of a transgene can be linked to a naïT-cell state by for example driving expression of transgene from a CD44 promoter. Expression of a transgene can be linked to a memory T-cell state by for example driving expression of transgene from a CD122 promoter. Expression of a transgene can be linked to a regulatory T-cell state by for example driving expression of transgene from a FOXP3 promoter etc.

CD4+ T-cell specific expression can be achieved by using −1076 to +20 (relative to the transcriptional start site) of the CD4 gene as a promoter. The DNA sequence of this promoter is shown as SEQ ID No. 1 below. Cloning this segment of the CD4 gene upstream of the transgene open-reading frame results in expression of the transgene whenever the CD4 gene is turned on within the T-cell. CD8+ specific expression can be achieved using an equivalent portion of the CD8 gene which is shown as SEQ ID No. 2 below.

(CD4 Promoter)
SEQ ID No. 1
AAGACAGGTTCTCACTCTGTCACTCAGGCTAGAGTGCAGTGGTGCAATCA
CGGTTCACTGCAGCCTCAACTTCCTGGGCTCAAGCGATCCCCCCACCTCG
GCCTCCTAAAATGCTGGGATTATAGGCATGAGCCACCACTCCCAGCCCCA
CTTTTTTCAGACTGGAAAACGCACACTCACATGTGCATCTTTAAATGATC
ACTTGGGCTGTGGTATGGAGAATGGCGACCAGTGAGGAGGCAGGAGCTGT
TGTCCGAGCAAGGGATGATATTGGCATCTTGGATTGGCATGGTGGCAGTA
GTGGTAGTGCAGAGTGACTTGGGTAGATTTTGGAGCCATTTAGAAGGTAA
CATCCACAGGAACTGGTAAATAAATACGTGGGAGAAGTTGGGTGAAGGGG
GTGTCAAAGATTACACCCAATTTATTTTGCTTGGGCAAGTTGGTGGATGG
TGAGCCCCTCACTGAGTGAGAAGCCTGGAGAAGCAGGTTTGGAGGGTGGT
AGTATGCAGGTGGTATGCATAGTTGGGGATGTGTGTTGAGTTTGCTATGT
CCGGTGAGCTTCCCAGTGGAGATGTCCAATGGGCAGACGGATACTCACAT
AGAGAGTTCATGGTAGATTCGGGCTAGAGGAAAGCACCTGAGGCCTGGCC
AGAGACGCCTAGAGGAACAGAGCCTGGTTAACAGTCACTCCTGGTGTCTC
AGATATTCTCTGCTCAGCCCACGCCCTCTCTTCCACACTGGGCCACCTAT
AAAGCCTCCACAGATACCCCTGGGGCACCCACTGGACACATGCCCTCAGG
GCCCCAGAGCAAGGAGCTGTTTGTGGGCTTACCACTGCTGTTCCCATATG
CCCCCAACTGCCTCCCACTTCTTTCCCCACAGCCTGGTCAGACATGGCGC
TACCACTAATGGAATCTTTCTTGCCATCTTTTTCTTGCCGCTTAACAGTG
GCAGTGACAGTTTGACTCCTGATTTAAGCCTGATTCTGCTTAACTTTTTC
CCTTGACTTTGGCATTTTCACTTTGACATGTTCCCTGAGAGCCTGGGGGG
TGGGGAACCCAGCTCCAGCTGGTGACGTTTGGGGCCGGCCCAGGCC
(CD8 Promoter)
SEQ ID No. 2
CACAGGAGGCTCAGCACTAATCGGTAGATACTGCGAGATGCTGGGAGGTT
AAGGGGCCTACCCGCAATATCTCTGGCCAATGCCTTGGGCTAGAAATGCC
ATAATTAGCCGCTCTTTTGATCCCTTGCAAAATGCGAATCCCACCGCACC
TCCACCCCACCCGAGTGGTAATCTCCTAGTGGTAATCTAAGTGAGCCTGT
GATAAGATAAGTAGCTCCTGGTGGTGAGGGTGAGAAATTGGGGAGCTGGA
GCCCCAGCCAGGGACGAGGCTGTAGGGGCTAGGGCGAAGATGGAGGCTGC
TGGGCCCCCAGATGGAAGACGGTAACGTGCGCCCGCTTCGTTTTTGCTCG
AGGTCAGTCAGGTGCAGACTGAATTCGAAGTCGCTCCCTCCTCCGCTCAA
CCCCGACCAGGCCAAAACTAAAGCAGCACGCCCCCTGCTGGGCCGACAGG
GCATCAGATTTTGCTGGACGCGGGTGACAGGCGAGATAGGGAGTGTCCCT
GCTGCTAGTGCCCCTGCTGCTAGTGCCTAGTTACCTGCA

Regulatory T-cell specific expression can be achieved by using a FOXP3 specific promoter. A promoter specific for FOXP3 is located in the region of −511 to +176 base pairs (relative to the transcriptional start site) of the FOXP3 gene. The DNA sequence of this promoter is shown as SEQ ID No. 3 below.

(FOXP3 Promoter)
SEQ ID No. 3
TCCCATCCACACATAGAGCTTCAGATTCTCTTTCTTTCCCCAGAGACCCT
CAAATATCCTCTCACTCACAGAATGGTGTCTCTGCCTGCCTCGGGTTGGC
CCTGTGATTTATTTTAGTTCTTTTCCCTTGTTTTTTTTTTTTCAAACTCT
ATACACTTTTGTTTTAAAAACTGTGGTTTCTCATGAGCCCTATTATCTCA
TTGATACCTCTCACCTCTGTGGTGAGGGGAAGAAATCATATTTTCAGATG
ACTCGTAAAGGGCAAAGAAAAAAACCCAAAATTTCAAAATTTCCGTTTAA
GTCTCATAATCAAGAAAAGGAGAAACACAGAGAGAGAGAAAAAAAAAACT
ATGAGAACCCCCCCCCACCCCGTGATTATCAGCGCACACACTCATCGAAA
AAAATTTGGATTATTAGAAGAGAGAGGTCTGCGGCTTCCACACCGTACAG
CGTGGTTTTTCTTCTCGGTATAAAAGCAAAGTTGTTTTTGATACGTGACA
GTTTCCCACAAGCCAGGCTGATCCTTTTCTGTCAGTCCACTTCACCAAGG
TGAGTGTCCCTGCTCTCCCCTACCAGATGTGGGCCCCATTGGAGGAGATG

Expression of a transgene can be linked to a naïT-cell state by for example driving expression of transgene from a CD44 promoter. A promoter specific for CD44 is located at CD44 at -908 to -118 from the transcriptional start site of the CD44 gene. The DNA sequence of this promoter is shown as SEQ ID No. 4 below.

(CD44 Promoter)
SEQ ID No. 4
GAAGTTGTATGGGAAGATGAATAGAAGAATAGGTGGTTGAATAAATTAA
AAGGTGTGTGGTTGGATGAATGAATGAGTGGGATGATAGATGGACCTAA
GTGGTTAGTGGATGGACAGGAGGATGGATGGATGTGAGAGCCCCAGAAG
GACATAAGGAAAGATGGGTGGATAGATGGATGGGCGGATGGAAGGATAT
TTAGGAGGATGAATGAGCATGTGTGTGGAGAGAGGTGCCCATTCACACT
GGCTTGAACACATGGGTTAGCTGAGCCAAATGCCAGCCCTATGACAGGC
CATCAGTAGCTTTCCCTGAGCTGTTCTGCCAAGAAGCTAAAATTCATTC
AAGCCATGTGGACTTGTTATTGAGGGGAAAAAGAATGAGCTCTCCCTCT
TTCCACTTGGAAGATTCACCAACTCCCCACCCCTCACTCCCCACTGTGG
GCACGGAGGCACTGCGCCACCCAGGGCAAGACCTCGCCCTCTCTCCAGC
TCCTCTCCCAGGATATCCAACATCCTGTGAAACCCAGAGATCTTGCTCC
AGCCGGATTCAGAGAAATTTAGCGGGAAAGGAGAGGCCAAAGGCTGAAC
CCAATGGTGCAA

Other markers for Naïve/central memory cells include: CCR7, CD62L, CD27, CD28, CD127. Promoters from these genes may be used to give naïve/central memory specific expression. The DNA sequences for CD27, CD28 and CD127 are shown below as SEQ ID No. 5, 6 and 7 respectively.

(CD27 promoter region)
SEQ ID No. 5
TTTTGTGGTGCTGGTTTCTGTATAAACCTGAAAAATTCTGAATTCCAAA
ACTTATCTGACCCCCAAAGTTTCAGATAAGAGCTTGTGGACCTGTGCTC
AATTCTGGTTCTCCTTCCTTCTTTCAACTGTTGTCTGTGAAAGGAGGGA
TGCAGGTATGGGAGACAGGAGTCCTGCGAATTCGTCTGTAAACTGTGGA
CGGGGGGGTGGGTGGGGGGGGGTAACGTGGGCACCTTTGTGCACAAGTG
CATGAATAGGAGGGGTGAGCAACTGTGTGTCCATCACCTTTTTGTCAAA
GAAGCAGGAGTCAGTGGGCTACGTGCTTCATGAGCAGGAGAGGCGGAAA
CTAAGGAAGGCTCATGTGTTGGAGGAAGCATGTTTGAAGAGCAGCAGGT
CTCACAGAGTTTGCTCTTTAATACTCTCCCCAGCACACGGAAGGGGAAG
GGGGTGGAGGTTGCTGCTATGAGAGAGAAAAAAAAAACAGCCACAATAG
AGATTCTGCCTTCAAAGGTTGGCTTGCCACCTGAAGCAGCCACTGCCCA
GGGGGTGCAAAGAAGAGACAGCAGCGCCCAGCTTGGAGGTGCTAACTCC
AGAGGCCAGCAT
(CD28 promoter region)
SEQ ID No. 6
CAGGTACCCACCATGATGCCTGGCTAATTTTTTGTATTTTCAATGGAGA
CGGGGTTTCACCATGTTGGCCAGGCTCGTCTTGACCTCCTGGCCTCAAA
TGATCCACCCACTTTGGCCTCCCAAATTGCTGGCATTACAGGCGTGAGC
CACTGCACCCGGCCTGTTCCTTCTTAAGAACACTTTGTCTCCCCTTTAA
TCTCTGCTGGATTTCAAGCACCCCTTTTACACAACTCTTGATATCCATC
AATAAAGAATAATTCCCATAAGCCCATCATGTAGTGACCGACTATTTTT
CAGTGACAAAAAAAAAGTCTTTAAAAATAGAAGTAAAAGTCTAAAGTCA
TCAAAACAACGTTATATCCTGTGTGAAATGCTGCAGTCAGGATGCCTTG
TGGTTTGAGTGCCTTGATCATGTGCCCTAAGGGGATGGTGGCGGTGGTG
GTGGCCGTGGATGACGGAGACTCTCAGGCCTTGGCAGGTGCGTCTTTCA
GTTCCCCTCACACTTCGGGTTCCTCGGGGAGGAGGGGCTGGAACCCTAG
CCCATCGTCAGGACAAAGATGCTCAGGCTGCTCTTGGCTCTCAACTTAT
TCCCTTCAATTC
(CD127 promoter region)
SEQ ID No. 7
CGAGACAAGCCTGGCCAACATGGCGAAACCCCGTCTCCACTGAAAACAC
AAAAATTAGGCTGGCATAGTGGCATTTGCCTGTAGTCCTAGCTACTCAG
GAGGCTGAGGCAGGAGAATTGCTTGAACCTGGGAGGTGGAAATTGCAGT
GAGCCGAGATCATGCTATTGTACTCCAGCCTGGGCAACAAAGCAAGACT
CTGTCTCAAAAAAATAAAAATTAAAAAAATAAAGTAGCCTCTAGCCTAA
GATAGCTTGAGCCTAGGTGTGAATCTACTGCCTTACTCTGATGTAAGCA
CAGTAAGTGTGGGGGCTGCAGGGAATATCCAGGAGGAACAATAATTTCA
GAGGCTCTGTCTCTTCATGTCCTTGACCTCTGCTTACAGCAGCAATACT
TTTACTCAGACTTCCTGTTTCTGGAACTTGCCTTCTTTTTTGCTGTGTT
TATACTTCCCTTGTCTGTGGTTAGATAAGTATAAAGCCCTAGATCTAAG
CTTCTCTGTCTTCCTCCCTCCCTCCCTTCCTCTTACTCTCATTCATTTC
ATACACACTGGCTCACACATCTACTCTCTCTCTCTATCTCTCTCAGAAT
GACAATTCTAGG

Other markers for terminally differentiated effector T cells include: CD57, KLRG1, CD161 (KLRB1), CD58 and CD122. Promoters from these genes may be used to give effector T cell specific expression. The DNA sequence for CD122 promoter is given as SEQ ID No. 8 below.

(CD122 promoter)
SEQ ID No. 8
TGCTAAACGGAGTAAGGGGCTTCCTGGAAGGCTGGGTGAAATGGGAGTCT
CGGAAAGATGGTGTGTTGCAGGCTGGGAGGAGGGTGAGACGCTGGGGTCA
CCTAGAGGGACCTGCTTGTGTGAAGCCTACGTATTAGTGGGTATGTGTGT
GACCGGATGGAGGCGTCAGAGGTGTTGGGTAGCCTGTGTGAGTTGGCGTG
GGGGTGATGTAGGAGGGGAGAGAGGGAGGGCCTGCGTTCCCTTGGCTCCT
GTGTGCAGCTAGGCCCCTATTTGACAATGTGTGTCTGTGTGTGTGTGTGT
GTGTGTGTGTGTGTGTGTGTGTGTGTGTGCCGCCCCCAGCGTAGGAGGCA
GATCTTTATCTGGCCCTGGGTGCTTGAGGAGTTTCAGGCTTTCTCATAAG
CCTCGTCTCCCCGCCTCTCCACCCCAGGCCTTGCCCCTCTATCCTCTGCA
CAGGAAGTGGGCTGGCTCTGGGCTTTTAGTCTTTGCGGCCCCAGCAGCCA
GAGCTCAGCAGGGCCCTGGAGAGATGGCCACGGTCCCAGCACCGGGGAGG
ACTGGAGAGCGCGCGCTGCCACCGCCCCATGTCTCAGCCAGGTGATGTCC

Several databases contain promoter sequence information. For example, EPDnew (Eukaryotic Promoter Database)—is a new collection of experimentally validated promoters in human (and other) genomes. (Reference: Dreos, R. et al. 2015. Nucl. Acids Res. 43 (D1):D92-D96).

Promoters which have not been described can be deduced by those skilled in the art. Briefly, deduction can be performed by analysis of genome sequences typically upstream of the transcriptional start site of gene in question. Comparisons with known motifs and other promoters can be made. Several public databases and software tools are available to assist with such analysis, for example:
Neural Network Promoter Prediction (Berkeley Drosophila Genome Project, U.S.A.)—dated (Reference: M.G. Reese 2001. Comput. Chem. 26: 51-6).
Promoter 2.0 Prediction Server (S. Knudsen,Center for Biological Sequence Analysis, Technical University of Denmark)—predicts transcription start sites of vertebrate Pol II promoters in DNA sequences
PROMOSER—Human, Mouse and Rat promoter extraction service (Boston University, U.S.A.)—maps promoter sequences and transcription start sites in mammalian genomes. (Reference: S. Anason et al. 2003. Nucl. Acids. Res. 2003 31: 3554-59).

Control Using miRNA Target Domains

A microRNA (miRNA) is a small non-coding RNA molecule (containing about 22 nucleotides) that functions in RNA silencing and post-transcriptional regulation of gene expression. miRNAs function via base-pairing with complementary sequences within mRNA molecules. As a result, these mRNA molecules are silenced, by one or more of the following processes: (i) cleavage of the mRNA strand into two pieces; (ii) destabilization of the mRNA through shortening of its poly(A) tail; and less efficient translation of the mRNA into proteins by ribosomes.

An alternative method to selectively control expression in the cotext of the present invention is the introduction of particular miRNA target sequences into the untranslated regions of a transcript. These miRNA target sequences direct destruction of the transcript by cognate miRNAs. The miRNA target sequences are selected so their cognate miRNA is expressed when expression of transgene is not desired. MicroRNAs are arguably most important in T cells during the earliest and last stages in T-cell biology. The first stages of early thymic differentiation have a crucial reliance on the microRNA network, while later stages and peripheral homeostasis are largely, although not completely, microRNA-independent. The most profound effects on T cells are in the activation of effector and regulatory functions of conventional and regulatory T cells, where microRNA deficiency results in a near-complete loss of function. The temporal activity of miRNA in T-cell differentiation is reviewed by Jeker, and Bluestone (2013; Immunol. Rev. 253, 65-81); Dooley et al (2013; Immunol. Rev. 253, 53-64) and Baumjohann and Ansel (2013; Nat. Rev. Immunol. 13: 666-678).

Appropriate miRNA target sequences can be selected by those skilled in the art from literature, databases and predictive software.

For example miRDB (Nathan Wong and Xiaowei Wang (2015) miRDB: an online resource for microRNA target prediction and functional annotations. Nucleic Acids Research. 43(D1):D146-152.)

A further example: microRNA.org. microRNA target predictions: The microRNA.org resource: targets and expression. Betel D, Wilson M, Gabow A, Marks DS, Sander C., Nucleic Acids Res. 2008 Jan; 36(Database Issue): D149-53.

Table 1 gives some examples of microRNA sequences important in T cells.

TABLE 1

| miRNA | Effect | Target sequence |
|---|---|---|
| miR-17 | Naïve T-cells | CCTATTCCAGCACTTTCAAGTAGCTGTGAT (SEQ ID No. 14) |
| miR-146 | Naïve T-cells | AGTTCAACAAAAGTTCTCACATGGAGTCCC (SEQ ID No. 15) |
| miR-214 | Activation | AACTTACCAAGGACAGGCAGGACCCCGTCC (SEQ ID No. 16) |
| miR-21 | Differentiation | TTTATTACTTTATTGGTGTTAAGGATAACA (SEQ ID No. 17) |
| | form naïve | |
| miR-181 | Differentiation | TGCTATGTAGATTTCTGAATGTGTTGTATT (SEQ ID No. 18) |
| | from naïve | |
| miR-9 | Differentiation | CCCTACCCCCCAACCCCTAGCCCAACCAAT (SEQ ID No. 19) |
| | from naive | |
| miR-29 | Polarization | CCTTTCACAT TGGTGCTTTTCCATTTATGC (SEQ ID No. 20) |
| miR-126 | Polarization | AAAGAGGTTTTTAATAATGAGGTCCTTCTG (SEQ ID No. 21) |
| miR-326 | Polarization | GTCTGCTATTCCCAGAGAGGTCTCAGAGGG (SEQ ID No. 22) |
| miR-155 | Regulatory | CTGCACTTATTGTAGGAAATTTTAATATAT (SEQ ID No. 23) |

Environmental Metabolites

In a second embodiment, the first aspect of the invention relates to a cell comprising an NOI which is selectively expressed by the cell depending on the presence of an environmental metabolite in the microenvironment of the cell.

The environmental metabolite may be a metabolite found in a tumour microenvironment. The metabolite may be directly or indirectly produced by the tumour.

Aryl Hydocarbon Receptor

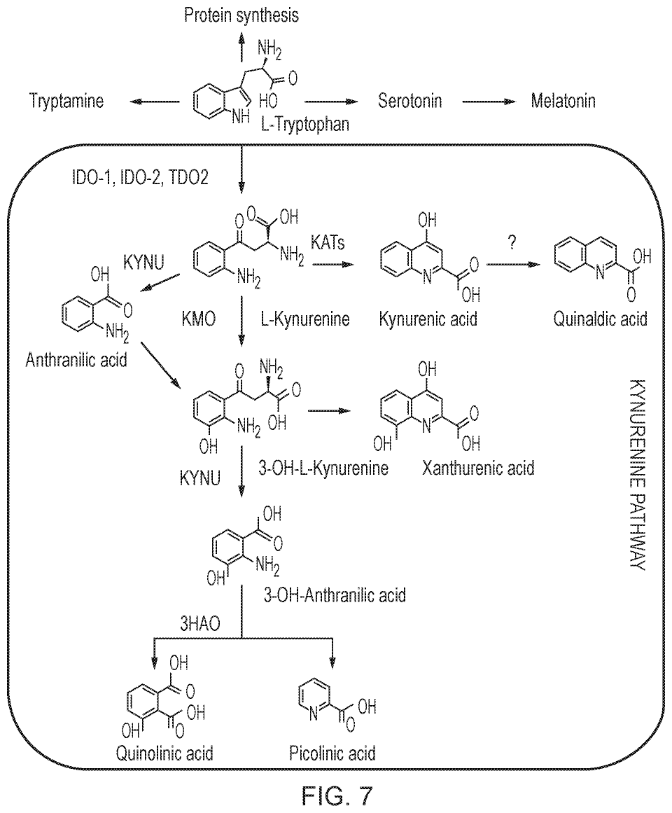

The cellular response to environmental toxins is mediated largely by activation of the Aryl Hydrocarbon Receptor (AHR). AHR activation occurs following binding of the toxin to a PAS (Per-Arnt-Sim) domain. This initiates structural changes resulting in release of cellular chaperones allowing dimerization with the ARNT transcription factor. Binding of the resulting AHR/ARNT heterodimer to specific DNA sequences (XRE—nobiotic recognition elements) results in the up-regulation of genes required to respond to the cellular insult (FIG. 6).

In the context of the present invention, the environmental metabolite may activate the aryl hydrocarbon receptor (AHR).

Expression of the nucleotide of interest may be upregulated by an AHR/ARNT heterodimer.

The nucleic acid sequence comprising the NOI may also comprise one or more xenobiotic recognition elements (XRE(s)) which are specifically recognised by the AHR/ARNT heterodimer.

The XRE core sequence is shown below as SEQ ID No. 12. This sequence is often contained within the consensus sequence shown as SEQ ID No. 13

(SEQ ID No. 12)
5′-GCGTG-3′
(SEQ ID No. 13)
5′-T/GNGCGTGA/CG/CA-3′

The nucleotide sequence of the present invention may comprise SEQ ID No. 12 or 13, together with an NOI. In the reverse orientation (i.e. the antisense strand), the XRE core sequence has the sequence CACGC (SEQ ID No. 24).

The nucleotide sequence of the invention may comprise SEQ ID No. 24. For example, an XRE promoter may comprise one of the following sequences:

(SEQ ID NO. 25)
CTGGTAAGCACGCCAATGAA,
or
(SEQ ID NO. 26)
TGAGTTCTCACGCTAGCAGATTGAGTTCTCACGCTAGCAGATTGAGTTCT
CACGCTAGCAGAT.

The Kynurenine Pathway

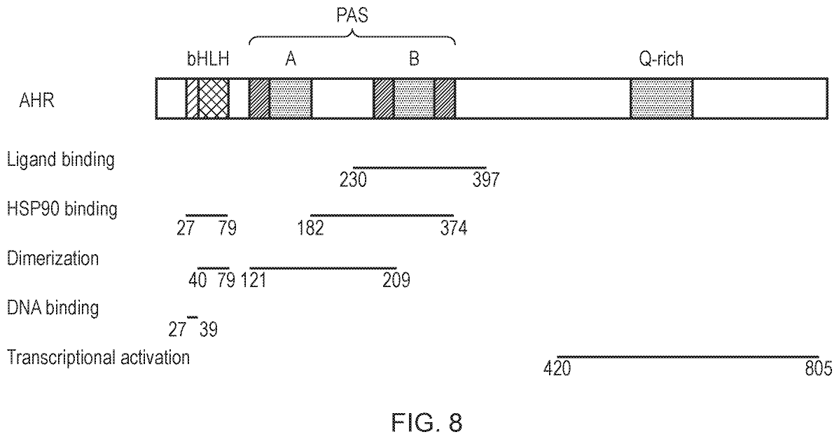

The tumour microenvironment, besides being a nutrient poor setting, also sustains a strong immunosuppressive activity, maintained in part by production of adenosine and of tryptophan metabolites within the microenvironment. The pathway of degradation of tryptophan to produce immunosuppressive products is shown in FIG. 7.

One of these metabolites, kynurenine acts by binding to the AHR and stimulating transcription via XRE sequences as shown schematically in FIG. 6.

In the context of the present invention, the environmental metabolite may be an adenosine or tryptophan metabolite. The environmental metabolite may, for example, be kynurenine, kynurenic acid, quinaldic acid, 3-OH-kyneurenine, xanthurenic acid, 3-OH-anthranilic acid, quinolic acid or picolinic acid. In particular, the environmental metabolite may be kynurenine.

Chimeric Antigen Receptor

The present invention provides a cell which comprises a chimeric antigen receptor (CAR) and a selectively expressed NOI.

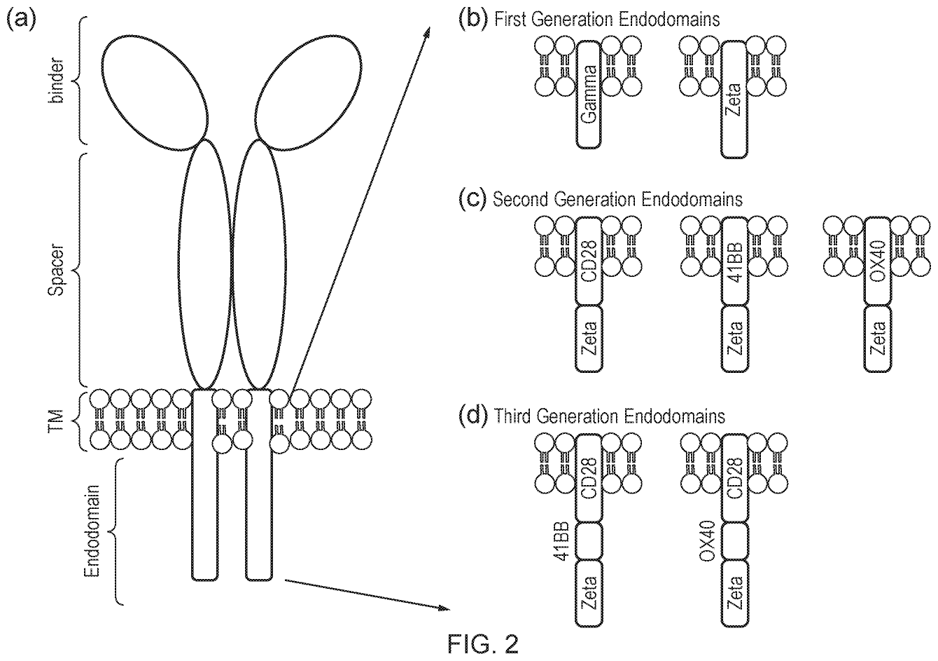

Classical CARs, which are shown schematically in FIG. 2, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8α even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41 BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 2. The antigen-binding domain used in the present invention may be a domain which is capable of binding a TAA as indicated therein.

TABLE 2

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The antigen-binding domain may comprise a proliferation-inducing ligand (APRIL) which binds to B-cell membrane antigen (BCMA) and transmembrane activator and calcium modulator and cyclophilin ligand interactor (TACI). A CAR comprising an APRIL-based antigen-binding domain is described in WO2015/052538.

Transmemebrane Domain

The transmembrane domain is the sequence of a classical CAR that spans the membrane. It may comprise a hydrophobic alpha helix. The transmembrane domain may be derived from CD28, which gives good receptor stability.

Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in FIG. 2B).

Car Component

In the cell of the present invention, the NOI may encode a CAR component.

For example, the NOI may encode a portion of a CAR, such as the intracellular signalling domain.

CAR signalling systems have previously been described which comprise two parts: a receptor component, which comprises the antigen binding domain, an optional spacer domain and the transmembrane domain; and an intracellular signalling component which comprises the intracellular signalling domain. One or more co-stimulatory domains may be located on the receptor component and/or the intracellular signalling component.

Heterodimerisation between the receptor component and the intracellular signalling component produces a functional CAR. Heterodimerisation may occur spontaneously, as described in WO2016/124930; or it may occur only in the presence of a chemical inducer of dimerization (CID), as described in WO2015/150771. In a third alternative, heterodimerization is disrupted by the presence of an agent, such as a particular small molecule, so CAR-mediated signalling only occurs in the absence of the agent. Such a system is described in WO2016/030691.

In the cell of the present invention, expression of a receptor component and/or an intracellular signalling component of such a CAR system may be selective, depending on the differentiation/exhaustion state of the cell or the presence of an environmental metabolite in the microenvironment of the cell. In other words the “CAR component” may be a receptor component or an intracellular signalling component.

It one particular embodiment, the cell may comprise an NOI encoding a receptor component under the control of a constitutively active promoter. For example, the cell may comprise two or more nucleic acids encoding intracellular signalling components with different co-stimulatory domains or co-stimulatory domain combinations each under the control of a different selective promoter/miRNA target. The co-stimulatory domain or co-stimulatory domain combination in the CAR system will therefore change with the differentiation or exhaustion state of the cell.

T-cell Receptor

The present invention also provides a cell which comprises an engineered T-cell receptor (TCR) and a selectively expressed NOI.

The TCR is a molecule expressed on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (a) chain and a beta (13) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vector. For example the genes for engineered TCRs may be reintroduced into autologous T cells and transferred back into patients for T cell adoptive therapies.

Nucleotide of Interest (NOI)

The cell of the present invention comprises a nucleotide of interest (NOI) which is selectively expressed depending on:
i) the differentiation/exhaustion state of the cell; or
ii) the presence of an environmental metabolite in the microenvironment of the cell.

The NOI may be RNA or DNA.

The NOI may encode a CAR, CAR component or TCR as described above.

The NOI may encode an agent which modulates CAR or TCR activity.

The NOI may encode an agent which modulates the activity of the CAR- or TCR-expressing cell.

The NOI may encode an agent which modulates the activity of the target cell.

The NOI may encode an agent which modulates the target cell microenvironment.

The cell may comprise two or more NOls which are selectively expressed depending on:
i) the differentiation/exhaustion state of the cell; or
ii) the presence of an environmental metabolite in the microenvironment of the cell. The cell may, for example produce a combination of agents which affect the CAR/TCR-expressing cell, the target cell, or the target cell microenvironment. The cell may, for example, produce a combination of cytokines or chemokines or a cytokine and a chemokine.

CAR/TCR Modulating Agent

The present invention also provides a cell which comprises a CAR or engineered TCR and an agent which modulates CAR or TCR activity. The agent may be selectively expressed depending on the transcriptional state of the cell.

The agent which modulates CAR/TCR activity may, for example, be a signal transduction modifying protein; a “dampener”; an inhibitory CAR or a cytokine signalling domain.

Signal Transduction Modifying Protein

WO2016/193696 describes various fusion proteins and truncated proteins which modulate the signalling pathways following immune cell activation.

The signal transduction modifying protein may, for example, be one of the following:
(i) a truncated protein which comprises an SH2 domain from a protein which binds a phosphorylated immunoreceptor tyrosine-based activation motif (ITAM), but lacks a kinase domain;
(ii) a truncated protein which comprises an SH2 domain from a protein which binds a phosphorylated immunoreceptor tyrosine-based inhibition motif (ITIM) but lacks a phosphatase domain;
(iii) a fusion protein which comprises (a) an SH2 domain from a protein which binds a phosphorylated immunoreceptor tyrosine-based activation motif (ITAM) or from a protein which binds a phosphorylated immunoreceptor tyrosine-based inhibition motif (ITIM); and (ii) a heterologous domain.

The signal transduction modifying protein may be a truncated protein which comprises a ZAP70 SH2 domain but lacks a ZAP70 kinase domain.

The signal transduction modifying protein may be a truncated protein which comprises an PTPN6 SH2 but lacks a PTPN6 phosphatase domain.

The signal transduction modifying protein may be a truncated protein which comprises a SHP-2 SH2 domain but lacks a SHP-2 phosphatase domain.

The signal transduction modifying protein may be a fusion protein which comprises (i) an SH2 domain from a protein which binds a phosphorylated immunoreceptor tyrosine-based activation motif (ITAM); and (ii) a phosphatase domain.

The fusion protein may, for example, comprise a ZAP70 SH2 domain, a PTPN6 or an SHP-2 phosphatase domain.

The signal transduction modifying protein may be a fusion protein which comprises (i) an SH2 domain from a protein which binds a phosphorylated immunoreceptor tyrosine-based inhibition motif (ITIM); and (ii) a kinase domain.

The fusion protein may comprise an SH2 domain from PTPN6 or SHP-2.

The fusion protein may comprise a Zap70 kinase domain

The fusion protein may comprise an AKT or JAK kinase domain.

The signal transduction modifying protein may be a fusion protein which comprises (i) an SH2 domain from a protein which binds a phosphorylated immunoreceptor tyrosine-based activation motif (ITAM) or from a protein which binds a phosphorylated immunoreceptor tyrosine-based inhibition motif (ITIM); and (ii) a heterologous signalling domain.

The fusion protein may comprise an SH2 domain from ZAP70, PTPN6 or SHP-2.

The heterologous signalling domain may be from a signalling molecule which is not usually activated by an ITAM or ITIM containing receptor.

The heterologous signalling domain may be a co-stimulatory domain. In this respect, the fusion protein may comprise a CD28, OX40 or 41 BB co-stimulatory domain.

The heterologous signalling domain may be an inhibitory domain. In this respect, the inhibitory domain may be or comprise the endodomain of CD148 or CD45. Alternatively, the heterologous signalling domain is or comprises the endodomain of ICOS, CD27, BTLA, CD30, GITR or HVEM.

The signal transduction modifying protein may be a fusion protein which comprises (i) an SH2 domain from a protein which binds a phosphorylated immunoreceptor tyrosine-based activation motif (ITAM); and (ii) an ITAM-containing domain.

The fusion protein may comprises a ZAP70 SH2 domain.

The ITAM-containing domain may be or comprise the endodomain of CD3-Zeta.

The signal transduction modifying protein may be a fusion protein which comprises (i) an SH2 domain from a protein which binds a phosphorylated immunoreceptor tyrosine-based inhibition motif (ITIM); and (ii) an ITIM-containing domain.

The fusion protein may comprise an SH2 domain from PTPN6 or SHP-2.

The ITIM-containing domain may be or comprise the endodomain from PD1, PDCD1, BTLA4, LILRB1, LAIR1, CTLA4, KIR2DL1, KIR2DL4, KIR2DL5, KIR3DL1 or KIR3DL3.

When the signal transduction modifying protein comprises a truncated protein which comprises a ZAP70 SH2 domain but lacks a ZAP70 kinase domain, the truncated protein may comprise or consist of the sequence shown as SEQ ID NO: 9.

ZAP70 complete SH2 domain
(SEQ ID NO: 9)
MPDPAAHLPFFYGSISRAEAEEHLKLAGMADGLFLLRQCLRSLGGYVLSL
VHDVRFHHFPIERQLNGTYAIAGGKAHCGPAELCEFYSRDPDGLPCNLRK
PCNRPSGLEPQPGVFDCLRDAMVRDYVRQTWKLEGEALEQAIISQAPQVE
KLIATTAHERMPWYHSSLTREEAERKLYSGAQTDGKFLLRPRKEQGTYAL
SLIYGKTVYHYLISQDKAGKYCIPEGTKFDTLWQLVEYLKLKADGLIYCL
KEACPNSSASNASGAAAPTLPAHPSTLTHP

ZAP70 has two SH2 domains at the N-terminal end of the sequence, at residues 10-102 and 163-254 of the sequence. The truncated protein or fusion protein of the invention may therefor comprise one or both of the sequences shown as SEQ ID No. 10 and 11.

ZAP70 SH2 1
(SEQ ID NO: 10)
FFYGSISRAEAEEHLKLAGMADGLFLLRQCLRSLGGYVLSLVHDVRFHHF
PIERQLNGTYAIAGGKAHCGPAELCEFYSRDPDGLPCNLRKPC
ZAP70 SH2 2
(SEQ ID NO: 11)
WYHSSLTREEAERKLYSGAQTDGKFLLRPRKEQGTYALSLIYGKTVYHYL
ISQDKAGKYCIPEGTKFDTLWQLVEYLKLKADGLIYCLKEAC

The fusion protein may comprise a variant of SEQ ID NO: 9, 10 or 11 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of CD3-zeta which allow the recruitment of ZAP70.

Dampener

In an alternative embodiment, the agent may be a phosphatase “damper” which causes dephosphorylation of the CAR or TCR endodomain, raising the threshold to activation in certain transcriptional states.

The dampener may be a membrane-tethered signal-dampening component (SDC) comprising a signal-dampening domain (SDD).

The SDD may be capable of inhibiting the intracellular signalling domain of the CAR.

The SDD may comprise a phosphatase domain capable of dephosphorylating immunoreceptor tyrosine-based activation motifs (ITAMs), for example the endodomain of CD148 or CD45 or the phosphatase domain of SHP-1 or SHP-2.

The SDD may comprise an immunoreceptor tyrosine-based inhibition motif (ITIM), for example the SDD may comprise an endodomain from one of the following inhibitory receptors: PD1, BTLA, 2B4, CTLA-4, GP49B, Lair-1, Pir-B, PECAM-1, CD22, Siglec 7, Siglec 9, KLRG1, ILT2, CD94-NKG2A and CD5.

The SDD may inhibits a Src protein kinase, such as Lck. The SDD may comprise the kinase domain of CSK.

The membrane-tethered SDC may, for example, comprise a transmembrane domain or a myristoylation sequence.

Inhibitory Car

The agent may be an inhibitory CAR, i.e. a CAR which comprises an inhibitory endodomain. The inhibitory endodomain may comprise a protein-tyrosine phosphatase (PTP), such as the PTP domain from SHP-1 or SHP-2.

Alternatively, the inhibitory endodomain may comprise an ITIM (Immunoreceptor Tyrosine-based Inhibition motif) containing endodomain such as that from CD22, LAIR-1, the Killer inhibitory receptor family (KIR), LILRB1, CTLA4, PD-1, BTLA etc. When phosphorylated, ITIMs recruits endogenous PTPN6 through its SH2 domain. If co-localised with an ITAM containing endodomain, dephosphorylation occurs and the activating CAR is inhibited.

Alternatively, the inhibitory CAR may comprise a phosphatase domain capable of dephosphorylating immunoreceptor tyrosine-based activation motifs (ITAMs), for example the endodomain of CD148 or CD45 or the phosphatase domain of SHP-1 or SHP-2.

Cytokine Signalling Domain

Many cell functions are regulated by members of the cytokine receptor superfamily. Signalling by these receptors depends upon their association with Janus kinases (JAKs), which couple ligand binding to tyrosine phosphorylation of signalling proteins recruited to the receptor complex. Among these are the signal transducers and activators of transcription (STATs), a family of transcription factors that contribute to the diversity of cytokine responses.

When a cytokine receptor binds its ligand, one or more of the following intracellular signaling pathways may be initiated:

(i) the JAK-STAT pathway

(ii) the MAP kinase pathway; and

(iii) the Phosphoinositide 3-kinase (P13K) pathway.

Cytokine receptors comprises an endodomain which causes “cytokine-type” cell signalling.

The agent of the present invention may be or comprise a cytokine receptor endodomain.

The endodomain may be derived from a type I cytokine receptor. Type I cytokine receptors share a common amino acid motif (WSXWS) in the extracellular portion adjacent to the cell membrane.

The endodomain may be derived from a type II cytokine receptor. Type II cytokine receptors include those that bind type I and type II interferons, and those that bind members of the interleukin-10 family (interleukin-10, interleukin-20 and interleukin-22).

Type I cytokine receptors include:

(i) Interleukin receptors, such as the receptors for IL-2, IL-3, IL-4, IL-5, IL-6, IL-7, IL-9, IL-11, IL-12, IL13, IL-15, IL-21, IL-23 and IL-27;

(ii) Colony stimulating factor receptors, such as the receptors for erythropoietin, GM-CSF, and G-CSF; and

(iii) Hormone receptor/neuropeptide receptor, such as hormone receptor and prolactin receptor

Members of the type I cytokine receptor family comprise different chains, some of which are involved in ligand/cytokine interaction and others that are involved in signal transduction. For example the IL-2 receptor comprises an α-chain, a β-chain and a γ-chain.

The IL-2 receptor common gamma chain (also known as CD132) is shared between the IL-2 receptor, IL-4 receptor, IL-7 receptor, IL-9 receptor, IL-13 receptor and IL-15 receptor.

CAR/TCR-Expressing Cell Modulating Agent

The NOI may encode an agent which modulates the activity of the CAR- or TCR-expressing cell.

For example, the agent may be a cytokine or chemokine, an adhesion molecule, or a transcription factor.

Cytokine/chemokine

The agent may be a cytokine or chemokine. For example be selected from: IL12, flexilL12, GM-CSF, IL7, ID 5, IL21, IL2 and CCL19. In particular, the agent may be IL-12.

Interleukin 12 (IL-12) is a potent immunomodulatory cytokine of particular interest for modulating the tumour microenvironment redirecting the immune response against cancer. IL-12 is systemically toxic therefore methods for producing IL-12 locally are of great interest. The method of the present invention provides a mechanism whereby an immunomodulatory cytokine may be produced in the presence of an environmental metabolite, such as kynurenine. Selective production of IL-12 in the presence of an metabolite such as kynurenine enables local production of IL-12 by the CAR- or TCR-expressing cell, only when it is present in the tumour microenvironment.

Adhesion Molecule

Cell adhesion molecules (CAMs) are proteins located on the cell surface involved in binding with other cells or with the extracellular matrix (ECM) in cell adhesion.

These proteins are typically transmembrane receptors and are composed of three domains: an intracellular domain that interacts with the cytoskeleton, a transmembrane domain, and an extracellular domain that interacts either with other CAMs of the same kind (homophilic binding) or with other CAMs or the extracellular matrix (heterophilic binding).

Most CAMs belong to four protein families: Ig (immunoglobulin) superfamily (IgSF CAMs), the integrins, the cadherins, and the selectins.

The agent of the present invention may be or comprise an adhesion molecule which modulates CAR- or TCR-expressing cell activity.

Transcription Factor

The agent of the invention may be or comprise a transcription factor which modulates activity of the CAR- or TCR-expressing cell.

A transcription factor is a protein which controls the rate of transcription of genetic information from DNA to messenger RNA, by binding to a specific DNA sequence and regulate the expression of a gene which comprises or is adjacent to that sequence.

Transcription factors work by promoting (as an activator), or blocking (as a repressor) the recruitment of RNA polymerase.

Transcription factors contain at least one DNA-binding domain (DBD), which attaches to either an enhancer or promoter region of DNA. Depending on the transcription factor, the transcription of the adjacent gene is either up- or down-regulated. Transcription factors also contain a trans-activating domain (TAD), which has binding sites for other proteins such as transcription coregulators.

Transcription factors use a variety of mechanisms for the regulation of gene expression, including stabilizing or blocking the binding of RNA polymerase to DNA, or catalyzing the acetylation or deacetylation of histone proteins. The transcription factor may have histone acetyltransferase (HAT) activity, which acetylates histone proteins, weakening the association of DNA with histones and making the DNA more accessible to transcription, thereby up-regulating transcription. Alternatively the transcription factor may have histone deacetylase (HDAC) activity, which deacetylates histone proteins, strengthening the association of DNA with histones and making the DNA less accessible to transcription, thereby down-regulating transcription. Another mechanism by which they may function is by recruiting coactivator or corepressor proteins to the transcription factor DNA complex.

The transcription may be constitutively active or conditionally active, i.e. requiring activation.

The transcription factor may be naturally occurring or artificial.

The transcription factor may increases the proportion of naï, central memory and/or stem-cell memory T cells in the CAR-T cell composition.

The transcription factor may, for example be a central memory repressing transcription factor such as BCL6 or BACH2. Central memory repressors inhibit the differentiation of T cells to effector memory cells, so that they remain as one of the less differentiated T-cell subtypes, such a naïand stem cell memory T-cells.

Alternatively that transcription factor may be an effector memory repressing transcription factor such as BLIMP-1.

Target Cell Modulating Agent

The NOI may encode an agent which modulates the activity of the target cell, for example, the tumour cell.

For example, the agent may be a toxin

The agent may be a toxin which is toxic to tumour cells. For example, the agent may be diphtheria toxin, pseudomonas toxin or shigella toxin.

Target Cell Microenvironment Modulating Agent

The NOI may encode an agent which modulates the environment of the target cell, for example, the tumour cell.

For example, the agent may be a cytokine such as IL-7 or IL-12 or a chemokine such as CCL19. Alternatively, the agent may affect the expression or activity of a cytokine or chemokine. For example, the agent may be a dominant negative version of a cytokine or chemokine. A dominant negative version may, for example, be a mutated or truncated version of the cytokine/chemokine which binds to the receptor and competes with the wild-type cytokine/chemokine but does not trigger cytokine/chemokine signalling.

For example, the agent may be a dominant negative version of a cytokine receptor or chemokine receptor. A dominant negative version may, for example, be a mutated or truncated version of the cytokine/chemokine receptor which binds to the cytokine blocking its binding to the wild-type cytokine/chemokine receptor.

Alternatively, the agent may be an antibody or antibody fragment which blocks or otherwise modulates a cytokine or chemokine signalling pathway.

Using Selective Expression to Optimise Cell Function

The nucleic acid sequence(s) or construct(s) of the invention may be designed to optimise cell function, for example by keeping cells in a naïve/undifferentiated state, reducing terminal differentiation or reducing exhaustion. Expression of one or more genes may be tailored to a particular T cell type, such as a CD4+, CD8+ or regulatory T cell.

For example, the cell may comprise one nucleic acid sequence which constitutively expresses a CAR or CAR component, but selectively expresses an inhibitory molecule, such as truncated ZAP70, a dampener or an inhibitory CAR. If the inhibitory molecule is expressed only when the T cell is exhausted, this will dampen down T cell activity and prevent further exhaustion.

The invention can also be used to tailor the co-stimulatory domains of a CAR to a particular differentiation state. For example, a CAR or CAR component comprising a CD28 co-stimulatory domain could be constitutively expressed, whereas a CAR or CAR component comprising an OX40 or 41 BB co-stimulatory domain may be expressed only when the cell is a differentiates to effector memory. In this way, the population dynamics are skewed to favour central memory/naïT-cells but upon differentiation rapid expansion is favoured.

Nucleic Acid Sequence

The present invention provides a nucleic acid sequence which comprises an NOI as described above.

The NOI may be under the control of a promoter which is selectively active depending on the differentiation/exhaustion state of the cell.

The nucleic acid may comprise a specific miRNA target sequence which causes transcript degradation at a certain differentiation/exhaustion state of the cell. The miRNA target sequence may, for example, bw in the 5′ untranslated region.

The nucleic acid sequence may comprise both a selectively active promoter and one or more miRNA target sequences as defined above.

The NOI may be under the control of a promoter which is selectively active depending the presence of an environmental metabolite in the microenvironment of the cell in which it is expressed.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Kit of Nucleic Acid Sequences

The present invention also provides a kit comprising two or more nucleic acid sequences, at least one of which is as defined above.

The kit may comprise one nucleic acid sequence under the control of a constitutively active promoter and one nucleic acid sequence under the control of a selectively active promoter.

The kit may comprise two nucleic acid sequences under the control of different selectively active promoters.

The kit may comprise two nucleic acid sequences, one which comprises a specific miRNA target sequence and one which doesn't.

The kit may comprise two nucleic acid sequences comprising different miRNA target sequences.

One or both nucleic acid sequences may comprise a combination of a selectively active promoter and an miRNA target sequence.

Nucleic Acid Construct

The present invention also provides a cassette or nucleic acid construct comprising two or more nucleic acid sequences, at least one of which is as defined above.

The nucleic acid construct may comprise one nucleic acid sequence under the control of a constitutively active promoter and one nucleic acid sequence under the control of a selectively active promoter.

The nucleic acid construct may comprise two nucleic acid sequences under the control of different selectively active promoters.

The nucleic acid construct may comprise two nucleic acid sequences, one which comprises a specific miRNA target sequence and one which doesn't.

The nucleic acid construct may comprise two nucleic acid sequences comprising different miRNA target sequences.

One or both nucleic acid sequences may comprise a combination of a selectively active promoter and an miRNA target sequence.

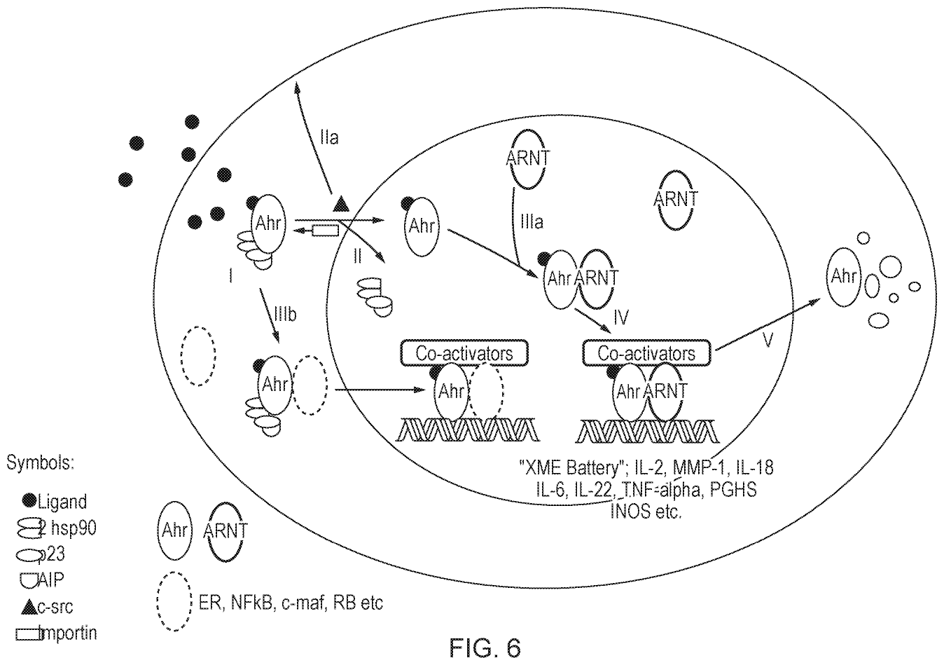

Expression cassettes can be engineered to incorporate split transcriptional systems. For example the vector can express two separate transcripts. In the arrangement shown in FIG. 5(b) a 5′ selectively active promoter drives transcription of a long transcript where the first open reading frame codes for a first protein which is selectively expressed. Downstream from this, a second constitutively active promoter in the same orientation as the first drives transcription of a shorter transcript where a second open reading frame codes for a second protein which is constitutively expressed. Both transcripts share the same polyA adenylation signal.

Alternatively, two separate promoters can drive expression of two independent transcripts. The transcripts may be oriented head-to-head as shown in FIG. 5(c) in which one transcript reads from the sense strand and the other reads from the anti-sense strand. Alternatively a constituitively active bi-directional promoter may be used as shown in FIG. 5(d) which results in transcription of two transcripts in opposite direction. Each transcript is controlled by a separate mlRNA target sequence.

T-cells can be engineered with combination of cassettes which have independent expression controlled either by promotors or miRNA target sequences, or both.

More conveniently, T-cells can be engineered with single cassettes which allow differential expression of different transgenes. For instance, a retroviral vector cassette can transcribe two transcripts one which is constitutively expressed and one which is conditionally expressed.

Specific promoters or miRNA target domains may on occasion provide insufficiently clean selective expression. Those skilled in the art can increase the complexity of the expression cassettes to increase selectiveness of expression. For instance a specific promoter and a specific miRNA targeting domain can be combined. Alternatively feed forward and feed back loops between different transcriptional units can be employed to tighten selectivity of expression.

Simple transcriptional switches offer good repression or activation. However, they often exhibit leakiness that precludes the gene of interest from being completely turned off or on. In some situations, this leakiness is acceptable to the required profile, but for some applications a tighter switch is needed. A transcriptional switch can be engineered to couple induced expression (selective promoter) with shRNA which acts against a constitutively active repressor which acts on inducible transcript. Such a system can be engineered so that induced expression is cleanly off/on and can be tuned to switch at precise levels of transcriptional activity (Deans et al (2007) Cell 130:363-372).

Vector/Kit of Vectors

The present invention also provides a vector, or kit of vectors which comprises one or more nucleic acid sequence(s) or nucleic acid construct(s) of the invention. Such a vector may be used to introduce the nucleic acid sequence(s) or construct(s) into a host cell so that it expresses the NOI.

The vector may, for example, be a plasmid or a viral vector, such as a retroviral vector or a lentiviral vector, or a transposon based vector or synthetic mRNA.

The vector may be capable of transfecting or transducing a cell.

Cell The cell of the present invention may be an immune effector cell, such as a T-cell or natural killer (NK) cell.

T or NK cells may be derived from a patient's own peripheral blood (1st party), or in the setting of a haematopoietic stem cell transplant from donor peripheral blood (2nd party), or peripheral blood from an unconnected donor (3rd party). T or NK cells may be activated and/or expanded prior to being transduced with nucleic acid encoding the molecules providing the CAR system according to the first aspect of the invention, for example by treatment with an anti-CD3 monoclonal antibody.

Alternatively, T or NK cells may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukopheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen

Method for Making Cell

CAR or TCR-expressing cells may be generated by introducing DNA or RNA coding for the CAR or TCR by one of many means including transduction with a viral vector, transfection with DNA or RNA.

The cell of the invention may be made by:
(i) isolation of a cell-containing sample from a subject or one of the other sources listed above; and
(ii) transduction or transfection of the cells with one or more a nucleic acid sequence(s) or nucleic acid construct as defined above in vitro or ex vivo.

The cells may then by purified, for example, selected on the basis of expression of the antigen-binding domain of the antigen-binding polypeptide.

Pharmaceutical Composition

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The present invention provides a cell of the present invention for use in treating and/or preventing a disease.

The invention also relates to the use of a cell of the present invention in the manufacture of a medicament for the treatment and/or prevention of a disease.

The disease to be treated and/or prevented by the methods of the present invention may be an infection, such as a viral infection.

The methods of the invention may also be for the control of pathogenic immune responses, for example in autoimmune diseases, allergies and graft-vs-host rejection.

The methods may be for the treatment of a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The CAR cells of the present invention may be capable of killing target cells, such as cancer cells. The target cell may be recognisable by expression of a TAA, for example the expression of a TAA provided above in Table 1.

The invention will now be further described by way of Examples, which are meant to serve to assist one of ordinary skill in the art in carrying out the invention and are not intended in any way to limit the scope of the invention.

### EXAMPLES

### Example 1

### Investigating Reporter Gene Expression Under the Control of Various Promoters in Different T Cell Subsets

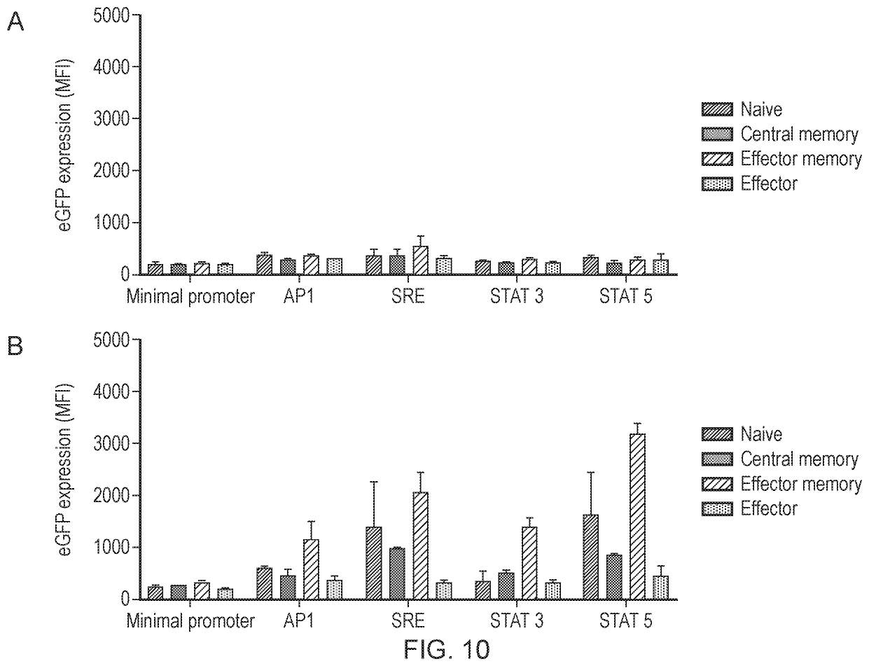

A self-inactivating retroviral vector was constructed in which an AP1/SRE/STAT3/STAT5 responsive promoter was cloned upstream of the coding sequence of the reporter gene eGFP. This first open-reading frame is followed by a PGK promoter and a second coding sequence encoding the RQR8 cell-surface marker. Primary human T cells from normal donors were transduced with the retroviral vector and either stimulated with 3 ug/mL PHA and 50 IL-2 U/mL for 72 hours or left unstimulated. The memory phenotype of the cells was analysed by flow cytometry and the results are shown in FIG. 9. The different memory compartments (naï, central memory, effector memory and effector) do not differ between the different transduced T cells after PHA and IL-2 stimulation.

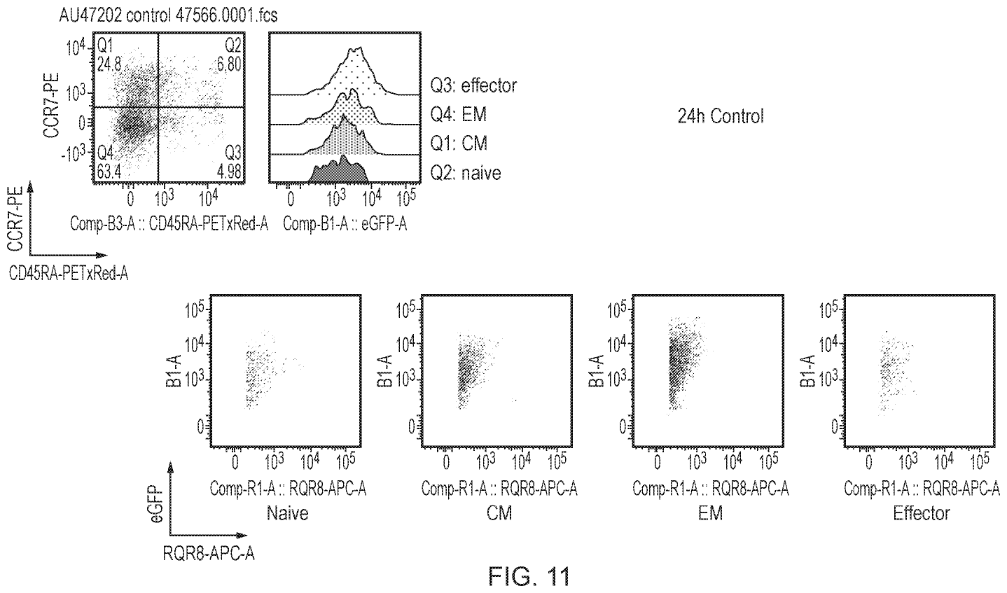

The eGFP expression levels of the different memory subsets was also analysed and the results are shown in FIG. 10. It was found that different response elements induced different patterns of eGFP upregulation depending on the memory subset: AP1 and STAT3-responsive promoters predominantly induced eGFP expression in the effector memory compartment whereas SRE and STAT5-responsive promoters showed eGFP upregulation in both naïand effector memory subsets.

### Example 2

### Investigating Reporter Gene Expression Under the Control of a CREB-Responsive Promoter in Different T Cell Subsets

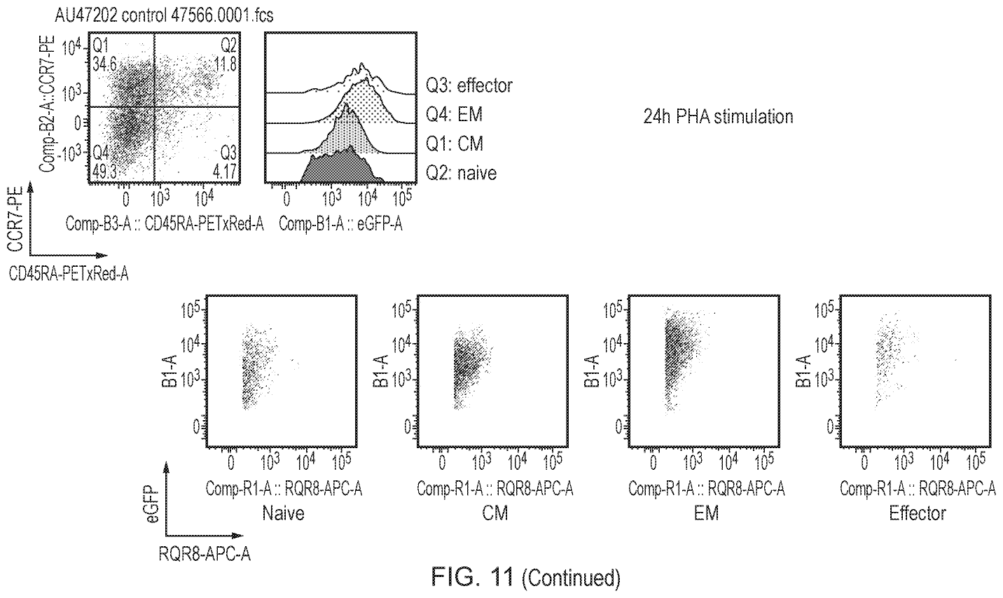

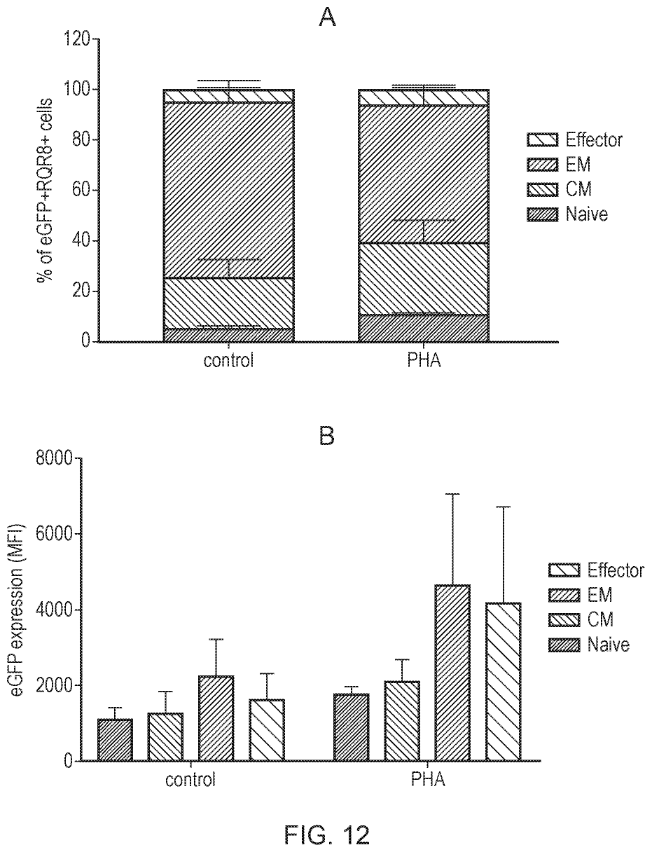

A self-inactivating retroviral vector was constructed in which an CREB responsive promoter was cloned upstream of the coding sequence of the reporter gene eGFP. This first open-reading frame is followed by a PGK promoter and a second coding sequence encoding the RQR8 cell-surface marker. Primary human T cells from normal donors were transduced with the retroviral vector and either stimulated with PHA for 24 hours or left unstimulated. The memory phenotype of the cells and eGFP expression was analysed by flow cytometry and the results are shown in FIGS. 11 and 12. The CREB-responsive promoter induced eGFP upregulation in the effector memory and effector cell subsets.

### Example 3

### Design and Construction of an Anti-CD19 CAR-T Cell with Differential Co-Stimulation in CD4+ T Cells

A self-inactivating retroviral vector is constructed whereby an initial promoter specific for CD4+ T-cells is cloned upstream of the coding sequence of a first CAR. This first CAR is constructed using the anti-CD19 scFv from fmc63, a CD8 spacer and a CD28− CD3Z endodomain. A PGK promoter is cloned downstream from this first coding sequence. A second coding sequence encoding a second CAR is cloned downstream from the PGK promoter. This second CAR is constructed using the anti-CD19 scFv from hd37, a CD8 spacer and a 41BB-CD3Z endodomain. This retroviral cassette should result in expression of the hd37/41 BB-CD3Z CAR in all cells, but in addition the fmc63/CD28-CD3Z CAR should be selectively expressed in CD4+ T-cells. T-cells are transduced with the retroviral vector. Primary human T-cells from normal donors are transduced with this retroviral vector. Differential expression of the two CARs is determined by flow cytometry. The use of two different scFvs against CD19 allows for verification of independent expression using two different anti-idiotype antibodies by flow cytometry. The performance of these T-cells is compared against T-cells transduced with simple vectors expressing either 41 BB-CD3Z or CD28-Z CARs in co-cultures in vitro and also in xenograft models of NALM6 into NSG mice.

### Example 4

### Design and Construction of an Anti-CD19 CAR-T Cell with Differential Co-Stimulation in naïand Central Memory T Cells

A self-inactivating retroviral vector is constructed whereby a CD127 specific promoter is cloned upstream of the coding sequence of a first CAR. This first CAR is constructed using the anti-CD19 scFv from fmc63, a CD8 spacer and a CD28-CD3Z endodomain. A PGK promoter is cloned downstream from this first coding sequence. A second coding sequence encoding a second CAR is cloned downstream from the PGK promoter. This second CAR is constructed using the anti-CD19 scFv from hd37, a CD8 spacer and a 41 BB-CD3Z endodomain. This retroviral cassette should result in expression of the hd37/41 BB-CD3Z CAR in all cells, but in addition the fmc63/CD28-CD3Z CAR should be selectively expressed in naïand central memory T-cells. T-cells are transduced with the retroviral vector. Primary human T-cells from normal donors are transduced with this retroviral vector. Differential expression of the two CARs is determined by flow cytometry. Use of two different scFvs against CD19 allowes for verification of independent expression using two different anti-idiotype antibodies by flow cytometry. The performance of these T-cells is compared against T-cells transduced with simple vectors expressing either 41 BB-CD3Z or CD28-Z CARs in co-cultures in vitro and also in xenograft models of NALM6 into NSG mice.

### Example 5

### Design and Construction of an Anti-CD19 CAR-T Cell with Differential Expression of IL-2 Depending on T Cell Differentiation State

A self-inactivating retroviral vector is constructed whereby an EOMES responsive promoter is cloned upstream of the coding sequence of a constitutively active IL2 construct. This first open-reading frame is followed by a PGK promoter. A second coding sequence encoding a CAR is cloned downstream from the PGK promoter. This CAR is constructed using the anti-CD19 scFv from hd37, a CD8 spacer and a 41 BB-CD3Z endodomain. This retroviral cassette should result in expression of the IL2 construct in differentiated T-cells, but not in naïor central memory T-cells. The CAR should be expressed in all T-cells. Primary human T-cells from normal donors are transduced with this retroviral vector. Differential expression of the two constructs is determined by flow cytometry. The performance of these T-cells is compared against T-cells transduced with simple vectors expressing either CAR alone or CAR with uncontrolled co-expression of IL2 construct in co-cultures in vitro and also in xenograft models of NALM6 into NSG mice.

### Example 6

### Design and Construction of a CAR-T Cell Sensitive to the Presence of Kynurenine

Kynurenine is an immunosuppressive metabolite synthesised from the amino acid tryptophan by the action of the enzyme IDO. Tumour-cell expressed IDO frequently leads to high levels of kynurenine within the microenvironment of solid tumours which in turn generates a highly immunosuppressive environment which may inhibit the function of tumour-reactive CAR T cells and prevent tumour rejection. Designing a mechanism by which CAR T cells can respond to the presence of kynurenine by expressing a desirable transgene allows these T cells to overcome kynurenine-mediated immunosuppression.

Retroviral constructs are generated consisting of the desired transgene under the control of a kynurenine-responsive promoter linked to of a marker of transduction, such as RQR8, under the control of a constitutively active promoter e.g. a PGK or EF1a promoter. Three kynurenine responsive transgene are investigated: a fluorescent marker protein, Green Fluorescent Protein (GFP); a CAR to a particular ligand, anti-CD19 CAR; and an enzyme, kynureninase, which prevents kynurenine from suppressing CAR T cell function.

In the case of GFP under the control of the kynurenine responsive promoter (SEQ ID No. 16), transduced T cells are cultured in kynurenine at concentrations of 0 uM (no kynurenine), 0.5 uM, 1 um, 2 uM, 5 uM, 10 uM, 20 uM and 50 uM for varying times including 0.5 hr, 1 hr, 2 hr, 4 hr, 6 hr, 12 hr and 24 hr. Kynurenine-induced expression of the GFP is measured by co-staining these cells at each timepoint for the transduction marker (RQR8) and assessing co-expression this this marker and GFP in kynurenine-treated cells compared to control cells which have not been exposed to kynurenine. The intensity of the GFP expression reflects the strength of the induction.

In the case of kynurenine-induced anti-CD19 CAR expression, transduced T cells are cultured in the presence of increasing amounts of kynurenine at concentrations of 0 uM (no kynurenine), 0.5 uM, 1 um, 2 uM, 5 uM, 10 uM, 20 uM and 50 uM for varying times including 0.5 hr, 1 hr, 2 hr, 4 hr, 6 hr, 12 hr and 24 hr. The kynurenine-induced expression of the CAR on the surface of the T cells is then measured in a functional assay by assessing CAR T cell responses. Transduced T cells are co-cultured with CD19+ Raji cells (a B-cell-derived tumour line) at T cell: target ratios of 4:1, 1:1 and 1:4 for 24 hrs and 72 hrs. These co-cultures are then stained for the T cell marker CD3, the transduction marker RQR8 and cell viability with a viability dye such as 7-AAD. Live target cells are identified by their lack of CD3 and RQR8 and their exclusion of 7-AAD. Live target cells are enumerated for each co-culture condition and compared to co-cultures with T cells which had not been exposed to kynurenine and so in which not CAR-mediated killing had taken place. Supernatants from these co-cultures would also be assessed for levels of the T cell cytokines IFN-gamma and IL2 by specific ELISA. Kynurenine-induced CAR expression would be expected to increase the levels of these cytokines in the co-culture supernatants as the expressed CARs would cause activation of the T cells in response to CD19-expressing targets.

In the case of kynureninase, a retroviral construct is generated consisting of kynureninase under the control of a kynurenine-responsive promoter (SEQ ID No. 16) linked to an anti-CD19 CAR under the control of a constitutively active promoter e.g. a PGK or EF1a promoter. Transduced T cells co-expressing the kynurenine-induced kynureninase and a CAR are co-cultured with CD19-expressing Raji cells in the presence of kynurenine at concentrations of 0 uM (no kynurenine), 0.5 uM, 1 um, 2 uM, 5 uM, 10 uM, 20 uM and 50 uM for 24 hrs or 72 hrs at T cell: target ratios of 4:1, 1:1 and 1:4. CAR-mediated killing of Raji cells is assessed at these timepoints as described above, together with the secretion of IFN-gamma and IL2. Kynurenine is expected to inhibit CAR function and this inhibition may be prevented upon the induction and expression of kynureninase.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which expresses a chimeric antigen receptor (CAR) or an engineered T-cell receptor (TCR), the cell comprising a nucleotide sequence of interest (NOI) which is selectively expressed by the cell depending on:
i) the differentiation/exhaustion state of the cell; or
ii) the presence of an environmental metabolite in the microenvironment of the cell.

2.-9. (canceled)

10. A cell according to claim 1 wherein the NOI is under the control of a selectively active promoter.

11. A cell according to claim 1 which comprises an miRNA target sequence such that expression of the NOI in the cell is controlled by an miRNA.

12.-15. (canceled)

16. A cell according to claim 1, wherein the NOI encodes a chimeric antigen receptor (CAR).

17.-19. (canceled)

20. A cell according to claim 1, wherein the NOI encodes an agent which modulates CAR or TCR activity.

21. (canceled)

22. A cell according to claim 1, wherein the NOI encodes an agent which modulates activity of the cell.

23. (canceled)

24. A cell according to claim 1, expressing a CAR or TCR which binds a target antigen on a target cell, wherein the NOI encodes an agent which modulates activity of the target cell.

25. (canceled)

26. A cell according to claim 1, expressing a CAR or TCR which binds a target antigen on a target cell, wherein the NOI encodes an agent which modulates the target cell microenvironment.

27. (canceled)

28. A nucleic acid sequence which comprises a nucleotide sequence of interest (NOI) under the control of a promoter which is selectively active depending on:
(i) the differentiation/exhaustion state of the cell in which it is expressed or
(ii) the presence of an environmental metabolite in the microenvironment of the cell in which it is expressed.

29. (canceled)

30. A nucleic acid sequence which comprises a nucleotide sequence of interest (NOI) and a specific miRNA target sequence which causes transcript degradation at a certain differentiation/exhaustion state of the cell in which the nucleic acid sequence is expressed.

31.-37. (canceled)

38. A nucleic acid construct, which comprises:
(i) a first nucleic acid sequence under the control of a constitutively active promoter; and
(ii) a second nucleic acid sequence under the control of a promoter which is selectively active depending on either the differentiation/exhaustion state of the cell in which it is expressed or the presence of an environmental metabolite in the microenvironment of the cell in which it is expressed.

39. (canceled)

40. A nucleic acid construct, which comprises:
(i) a first nucleic acid sequence which comprises a specific miRNA target sequence which causes transcript degradation at a certain differentiation/exhaustion state of the cell in which the nucleic acid construct is expressed; and
(ii) a second nucleic acid sequence which lacks a specific miRNA target sequence.

41. (canceled)

42.-46. (canceled)

47. A vector which comprises a nucleic acid sequence according to claim 28.

48. A method for making a cell according to claim 1 which comprises the step of introducing into a cell:
(i) a vector which comprises a nucleic acid sequence which comprises a nucleotide sequence of interest (NOI) under the control of a promoter which is selectively active depending on:
(a) the differentiation/exhaustion state of the cell in which it is expressed or
(b) the presence of an environmental metabolite in the microenvironment of the cell in which it is expressed;
(ii) a vector which comprises a nucleic acid sequence which comprises a nucleotide sequence of interest (NOI) under the control of a promoter which is selectively active depending the presence of an environmental metabolite in the microenvironment of the cell in which it is expressed; or
(iii) a vector which comprises a nucleic acid sequence which comprises a nucleotide sequence of interest (NOI) and a specific miRNA target sequence which causes transcript degradation at a certain differentiation/exhaustion state of the cell in which the nucleic acid sequence is expressed.

49. (canceled)

50. A pharmaceutical composition comprising a plurality of cells according to claim 1.

51. (canceled)

52. A method for treating cancer, which comprises the step of administering a pharmaceutical composition according to claim 50 to a subject.

53.-55. (canceled)

56. A vector which comprises a nucleic acid sequence according to claim 30.


SEQUENCE LISTING (32 sequences)

SEQ ID NO: 1 - DNA, 1096 bp, Artificial Sequence
  CD4 promoter
aagacaggttctcactctgtcactcaggctagagtgcagtggtgcaatcacggttcactgcagcctcaacttcctgggctcaagcgatccccccacctcggcctcctaaaatgctgggattataggcatgagccaccactcccagccccacttttttcagactggaaaacgcacactcacatgtgcatctttaaatgatcacttgggctgtggtatggagaatggcgaccagtgaggaggcaggagctgttgtccgagcaagggatgatattggcatcttggattggcatggtggcagtagtggtagtgcagagtgacttgggtagattttggagccatttagaaggtaacatccacaggaactggtaaataaatacgtgggagaagttgggtgaagggggtgtcaaagattacacccaatttattttgcttgggcaagttggtggatggtgagcccctcactgagtgagaagcctggagaagcaggtttggagggtggtagtatgcaggtggtatgcatagttggggatgtgtgttgagtttgctatgtccggtgagcttcccagtggagatgtccaatgggcagacggatactcacatagagagttcatggtagattcgggctagaggaaagcacctgaggcctggccagagacgcctagaggaacagagcctggttaacagtcactcctggtgtctcagatattctctgctcagcccacgccctctcttccacactgggccacctataaagcctccacagatacccctggggcacccactggacacatgccctcagggccccagagcaaggagctgtttgtgggcttaccactgctgttcccatatgcccccaactgcctcccacttctttccccacagcctggtcagacatggcgctaccactaatggaatctttcttgccatctttttcttgccgcttaacagtggcagtgacagtttgactcctgatttaagcctgattctgcttaactttttcccttgactttggcattttcactttgacatgttccctgagagcctggggggtggggaacccagctccagctggtgacgtttggggccggcccaggcc

SEQ ID NO: 2 - DNA, 540 bp, Artificial Sequence
  CD8 promoter
cacaggaggctcagcactaatcggtagatactgcgagatgctgggaggttaaggggcctacccgcaatatctctggccaatgccttgggctagaaatgccataattagccgctcttttgatcccttgcaaaatgcgaatcccaccgcacctccaccccacccgagtggtaatctcctagtggtaatctaagtgagcctgtgataagataagtagctcctggtggtgagggtgagaaattggggagctggagccccagccagggacgaggctgtaggggctagggcgaagatggaggctgctgggcccccagatggaagacggtaacgtgcgcccgcttcgtttttgctcgaggtcagtcaggtgcagactgaattcgaagtcgctccctcctccgctcaaccccgaccaggccaaaactaaagcagcaccgccccctgctgggccgacagggcatcagattttgctggacgcgggtgacaggcgagatagggagtgtccctgctgctagtgcccctgctgctagtgcctagttacctgca

SEQ ID NO: 3 - DNA, 600 bp, Artificial Sequence
  FOXP3 promoter
tcccatccacacatagagcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccccccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaaggtgagtgtccctgctctcccctaccagatgtgggccccattggaggagatg

SEQ ID NO: 4 - DNA, 600 bp, Artificial Sequence
  CD44 promoter
gaagttgtatgggaagatgaatagaagaataggtggttgaataaattaaaaggtgtgtggttggatgaatgaatgagtgggatgatagatggacctaagtggttagtggatggacaggaggatggatggatgtgagagccccagaaggacataaggaaagatgggtggatagatggatgggcggatggaaggatatttaggaggatgaatgagcatgtgtgtggagagaggtgcccattcacactggcttgaacacatgggttagctgagccaaatgccagccctatgacaggccatcagtagctttccctgagctgttctgccaagaagctaaaattcattcaagccatgtggacttgttattgaggggaaaaagaatgagctctccctctttccacttggaagattcaccaactccccacccctcactccccactgtgggcacggaggcactgcgccacccagggcaagacctcgccctctctccagctcctctcccaggatatccaacatcctgtgaaacccagagatcttgctccagccggattcagagaaatttagcgggaaaggagaggccaaaggctgaacccaatggtgcaa

SEQ ID NO: 5 - DNA, 600 bp, Artificial Sequence
  CD27 promoter region
ttttgtggtgctggtttctgtataaacctgaaaaattctgaattccaaaacttatctgacccccaaagtttcagataagagcttgtggacctgtgctcaattctggttctccttccttctttcaactgttgtctgtgaaaggagggatgcaggtatgggagacaggagtcctgcgaattcgtctgtaaactgtggacgggggggtgggtgggggggggtaacgtgggcacctttgtgcacaagtgcatgaataggaggggtgagcaactgtgtgtccatcacctttttgtcaaagaagcaggagtcagtgggctacgtgcttcatgagcaggagaggcggaaactaaggaaggctcatgtgttggaggaagcatgtttgaagagcagcaggtctcacagagtttgctctttaatactctccccagcacacggaaggggaagggggtggaggttgctgctatgagagagaaaaaaaaaacagccacaatagagattctgccttcaaaggttggcttgccacctgaagcagccactgcccagggggtgcaaagaagagacagcagcgcccagcttggaggtgctaactccagaggccagcat

SEQ ID NO: 6 - DNA, 600 bp, Artificial Sequence
  CD28 promoter region
caggtacccaccatgatgcctggctaattttttgtattttcaatggagacggggtttcaccatgttggccaggctcgtcttgacctcctggcctcaaatgatccacccactttggcctcccaaattgctggcattacaggcgtgagccactgcacccggcctgttccttcttaagaacactttgtctcccctttaatctctgctggatttcaagcaccccttttacacaactcttgatatccatcaataaagaataattcccataagcccatcatgtagtgaccgactatttttcagtgacaaaaaaaaagtctttaaaaatagaagtaaaagtctaaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattc

SEQ ID NO: 7 - DNA, 600 bp, Artificial Sequence
  CD127 promoter region
cgagacaagcctggccaacatggcgaaaccccgtctccactgaaaacacaaaaattaggctggcatagtggcatttgcctgtagtcctagctactcaggaggctgaggcaggagaattgcttgaacctgggaggtggaaattgcagtgagccgagatcatgctattgtactccagcctgggcaacaaagcaagactctgtctcaaaaaaataaaaattaaaaaaataaagtagcctctagcctaagatagcttgagcctaggtgtgaatctactgccttactctgatgtaagcacagtaagtgtgggggctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcagaatgacaattctagg

SEQ ID NO: 8 - DNA, 600 bp, Artificial Sequence
  CD122 promoter
tgctaaacggagtaaggggcttcctggaaggctgggtgaaatgggagtctcggaaagatggtgtgttgcaggctgggaggagggtgagacgctggggtcacctagagggacctgcttgtgtgaagcctacgtattagtgggtatgtgtgtgaccggatggaggcgtcagaggtgttgggtagcctgtgtgagttggcgtgggggtgatgtaggaggggagagagggagggcctgcgttcccttggctcctgtgtgcagctaggcccctatttgacaatgtgtgtctgtgtgtgtgtgtgtgtgtgtgtgtgtgtgtgtgtgtgtgtgtgccgcccccagcgtaggaggcagatctttatctggccctgggtgcttgaggagtttcaggctttctcataagcctcgtctccccgcctctccaccccaggccttgcccctctatcctctgcacaggaagtgggctggctctgggcttttagtctttgcggccccagcagccagagctcagcagggccctggagagatggccacggtcccagcaccggggaggactggagagcgcgcgctgccaccgccccatgtctcagccaggtgatgtcc

SEQ ID NO: 9 - protein, 280 aa, Artificial Sequence
  ZAP70 complete SH2 domain
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHP

SEQ ID NO: 10 - protein, 93 aa, Artificial Sequence
  ZAP70 SH2 1
FFYGSISRAEAEEHLKLAGMADGLFLLRQCLRSLGGYVLSLVHDVRFHHFPIERQLNGTYAIAGGKAHCGPAELCEFYSRDPDGLPCNLRKPC

SEQ ID NO: 11 - protein, 92 aa, Artificial Sequence
  ZAP70 SH2 2
WYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 12 - DNA, 5 bp, Artificial Sequence
  xenobiotic recognition elements (XRE(s)) core sequence
gcgtg

SEQ ID NO: 13 - DNA, 10 bp, Artificial Sequence
  XRE consensus sequence
  misc_feature (2)..(2): n is a, c, g, or t
kngcgtgmsa

SEQ ID NO: 14 - DNA, 30 bp, Artificial Sequence
  microRNA target sequence, miR-17
cctattccagcactttcaagtagctgtgat

SEQ ID NO: 15 - DNA, 30 bp, Artificial Sequence
  microRNA target sequence, miR-146
agttcaacaaaagttctcacatggagtccc

SEQ ID NO: 16 - DNA, 30 bp, Artificial Sequence
  microRNA target sequence, miR-214
aacttaccaaggacaggcaggaccccgtcc

SEQ ID NO: 17 - DNA, 30 bp, Artificial Sequence
  microRNA target sequence, miR-21
tttattactttattggtgttaaggataaca

SEQ ID NO: 18 - DNA, 30 bp, Artificial Sequence
  microRNA target sequence, miR-181
tgctatgtagatttctgaatgtgttgtatt

SEQ ID NO: 19 - DNA, 30 bp, Artificial Sequence
  microRNA target sequence, miR-9
ccctaccccccaacccctagcccaaccaat

SEQ ID NO: 20 - DNA, 30 bp, Artificial Sequence
  microRNA target sequence, miR-29
cctttcacattggtgcttttccatttatgc

SEQ ID NO: 21 - DNA, 30 bp, Artificial Sequence
  microRNA target sequence, miR-126
aaagaggtttttaataatgaggtccttctg

SEQ ID NO: 22 - DNA, 30 bp, Artificial Sequence
  microRNA target sequence, miR-326
gtctgctattcccagagaggtctcagaggg

SEQ ID NO: 23 - DNA, 30 bp, Artificial Sequence
  microRNA target sequence, miR-155
ctgcacttattgtaggaaattttaatatat

SEQ ID NO: 24 - DNA, 5 bp, Artificial Sequence
  XRE core sequence
cacgc

SEQ ID NO: 25 - DNA, 20 bp, Artificial Sequence
  XRE promoter
ctggtaagcacgccaatgaa

SEQ ID NO: 26 - DNA, 63 bp, Artificial Sequence
  XRE promoter
tgagttctcacgctagcagattgagttctcacgctagcagattgagttctcacgctagcagat

SEQ ID NO: 27 - DNA, 48 bp, Artificial Sequence
  AP-1 responsive promoter
tgagtcagtgactcagtgagtcagtgactcagtgagtcagtgactcag

SEQ ID NO: 28 - DNA, 87 bp, Artificial Sequence
  CREB responsive promoter
gcaccagacagtgacgtcagctgccagatcccatggccgtcatactgtgacgtctttcagacaccccattgacgtcaatgggagaac

SEQ ID NO: 29 - DNA, 115 bp, Artificial Sequence
  SRE responsive promoter
aggatgtccatattaggacatctaggatgtccatattaggacatctaggatgtccatattaggacatctaggatgtccatattaggacatctaggatgtccatattaggacatct

SEQ ID NO: 30 - DNA, 120 bp, Artificial Sequence
  TCF-LEF responsive promoter
agatcaaagggtttaagatcaaagggcttaagatcaaagggtataagatcaaagggcctaagatcaaagggactaagatcaaagggtttaagatcaaagggcttaagatcaaagggccta

SEQ ID NO: 31 - DNA, 125 bp, Artificial Sequence
  STAT3 responsive promoter
agcttcatttcccgtaaatcgtcgaagcttcatttcccgtaaatcgtcgaagcttcatttcccgtaaatcgtcgaagcttcatttcccgtaaatcgtcgaagcttcatttcccgtaaatcgtcga

SEQ ID NO: 32 - DNA, 75 bp, Artificial Sequence
  STAT5 responsive promoter
agttctgagaaaagtagttctgagaaaagtagttctgagaaaagtagttctgagaaaagtagttctgagaaaagt